(19) United States
(12) Patent Application Publication
(10) Pub. No.: US 2017/0087167 A1
(43) Pub. Date: Mar. 30, 2017

(54) MODIFICATION OF DRUGS FOR INCORPORATION INTO NANOPARTICLES

(71) Applicant: UNIVERSITY OF GEORGIA RESEARCH FOUNDATION, INC., Athens, GA (US)

(72) Inventors: Akil Abraham Kalathil, McDonough, GA (US); Bhabatosh Banik, Athens, GA (US); Anil Kumar, Athens, GA (US); Shanta Dhar, Miami, FL (US)

(21) Appl. No.: 15/280,010

(22) Filed: Sep. 29, 2016

Related U.S. Application Data
(60) Provisional application No. 62/234,591, filed on Sep. 29, 2015.

(51) Int. Cl. A61K 31/621; A61K 47/48; A61K 9/16

(52) U.S. Cl. CPC A61K 31/621; A61K 9/16; A61K 47/48038

(57) ABSTRACT

Aspirin is chemically modified to generate a prodrug that releases aspirin in cellular milieu. The prodrug has a lipophilic tail that may enhance uptake efficiency in nanoparticles. Nanoparticles including the prodrugs may be effective for treating inflammatory disorders, including neurodegenerative disorders.

### RELATED APPLICATIONS

This application claims the benefit of U.S. Provisional Patent Application No. 62/234,591 filed on Sep. 29, 2015, which application is hereby incorporated herein by reference in its entirety.

### STATEMENT OF GOVERNMENTAL SUPPORT

This invention was made with government support under grant number W81XWH-12-1-0406, awarded by the Department of Defense of the United States government; and under grant number R01NS093314, awarded by the National Institute of Neurological Disorders and Stroke of National Institutes of Health. The government has certain rights in the invention.

### FIELD

The present disclosure relates to modification of aspirin that can enhance loading into nanoparticles, nanoparticles containing modified aspirin, and methods of use thereof.

### BACKGROUND AND INTRODUCTION

Aspirin may be an important therapeutic additive for neurodegenerative diseases. However, its physiochemical properties may prevent adequate delivery to tissue.

Nanoparticles can aid in improved delivery of aspirin to target tissues. However, aspirin may not be incorporated into nanoparticles, particularly nanoparticles having a hydrophobic core, at sufficiently high levels.

### SUMMARY

The present disclosure describes, among other things, chemical modification of a non-steroidal anti-inflammatory drug (NSAID), such as aspirin, to generate a prodrug having a lipophilic moiety. It is believed that the lipophilic moiety can enhance loading efficiency of the NSAID prodrug into nanoparticles, particularly nanoparticles having a hydrophobic core. In various embodiments disclosed herein, the NSAID prodrug is incorporated into nanoparticles of a size and nature that have been previously shown to accumulate in the brain.

In various embodiments disclosed herein an NSAID prodrug has the following structure:

(T)ₙ−L−(D)ₘ (I),

where T is a lipophilic moiety; n is a positive integer (such as 1 to 10); D is a releasable anti-inflammatory agent moiety; m is a positive integer (such as 1 to 50); and L is a linker. In some embodiments, T is a saturated or unsaturated, straight or branched chain hydrocarbon having between 4 and 20 carbons. In some embodiments, T is a straight or branched chain C₄-C₂₀ alkyl. In some embodiments, T is octanyl. In some embodiments, n is 1. In some embodiments, D is bound to L via an ester linkage. In some embodiments, m is 2 or more. In some embodiments, m is 4. In some embodiments, L is a 2, 2, bis(methoxy)propionyl moiety.

In various embodiments, the releasable NSAID moiety is a releasable aspirin moiety.

In various embodiments disclosed herein an aspirin prodrug has the following structure:

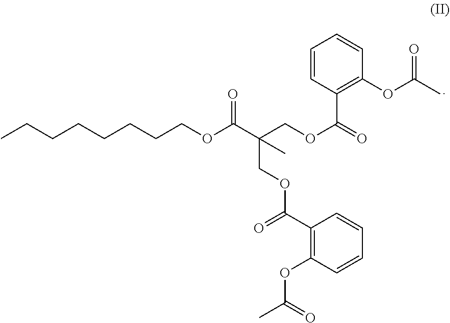 [SMILES: CCCCCCCCOC(=O)C(C)(COC(=O)c1ccccc1OC(C)=O)COC(=O)c1ccccc1OC(C)=O]

In various embodiments disclosed herein an aspirin prodrug has the following structure:

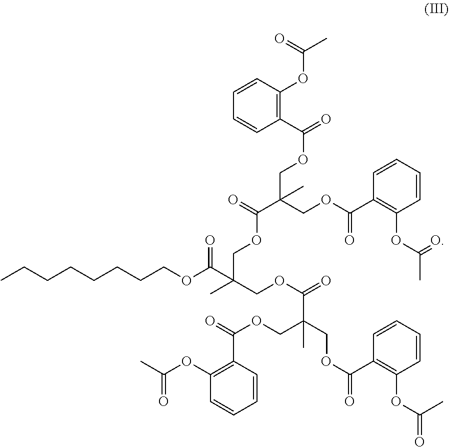 [SMILES: CCCCCCCCOC(=O)C(C)(COC(=O)C(C)(COC(=O)c1ccccc1OC(C)=O)COC(=O)c1ccccc1OC(C)=O)COC(=O)C(C)(COC(=O)c1ccccc1OC(C)=O)COC(=O)c1ccccc1OC(C)=O]

As indicted above, the NSAID prodrugs, such as the aspirin prodrugs, described herein can be incorporated into nanoparticles. Preferably, the nanoparticle has a hydrophobic core. In some embodiments, the nanoparticle has a mitochondria targeting moiety. In some embodiments, the nanoparticle has a diameter of 150 nanometers or less, such as 100 nanometers or less. Nanoparticles having such diametric dimensions may be better able to cross the blood brain barrier.

The nanoparticles described herein can be administered to patients in need thereof. Because the nanoparticles include a NSAID prodrug such as an aspirin prodrug, the nanoparticles can be used to inhibit cyclooxygenase. In some embodiments, the nanoparticles can be used to treat an inflammatory disease. In some embodiments the nanoparticles can be used to treat a neurodegenerative disease.

Advantages of one or more of the various embodiments presented herein over prior therapies including an NSAID will be readily apparent to those of skill in the art based on the following detailed description when read in conjunction with the accompanying drawings.

### BRIEF DESCRIPTION OF THE DRAWINGS

FIG. 1A is a schematic drawing illustrating the structure of two newly constructed hydrophobic aspirin derivatives.

FIG. 1B (top and bottom panels) provides graphs of analyses of physicochemical properties of NPs including hydrophobic Oc-[G1]-(Asp)₂ and Oc-[G2]-(Asp)₄. In the top panel the average zeta potential is shown. In the bottom panel, the average diameter is shown.

FIG. 1C (top and bottom panels) provides graphs of cytotoxic properties of these NPs in RAW 264.7 macrophages as determined by the MTT assay. Top panel: Oc-[G1]-(Asp)₂, targeted and non-target nanoparticles; Bottom panel Oc-[G1]-(Asp)₄, targeted and non-target nanoparticles.

FIG. 2A is a schematic drawing of the structure of Oc-[G2]-(Asp)₄.

FIG. 2B (top and bottom panels) are schematic drawings of synthesis schemes for targeted (bottom) and non-targeted (top) (Asp)₄-NPs from different polymers and Oc-[G2]-(Asp)₄.

FIG. 2C are graphs (top left, top right, middle left, middle right) and images (lower left, lower right) of data associated with (Asp)₄-NPs. Diameter (top left), zeta potential (top right), percent loading (middle left), % EE (middle right), and TEM of targeted (bottom right) and non-targeted (bottom left) (Asp)₄-NPs are shown.

FIG. 2D is a graph showing release kinetics of Oc-[G2]-(Asp)₄ from T and NT-NPs.

FIG. 3A is a schematic drawing of an experimental design for evaluation of anti-inflammatory properties of Oc-[G2]-(Asp)₄ and its NP under preventative condition using BALB/c Albino male mice.

FIG. 3B (top, middle and bottom panels) are graphs showing pro-inflammatory TNF-α (top), IL-6 (middle) and anti-inflammatory IL-10 (bottom) levels in the serum samples of BALB/c Albino mice treated with different constructs and LPS. ***: P<0.001; **: P=0.001-0.01; ns: non-significant.

The schematic drawings in are not necessarily to scale.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

The present disclosure describes, among other things, chemical modification of NSAIDs such as aspirin to generate a prodrug having a lipophilic moiety. It is believed that the lipophilic moiety can enhance loading efficiency of the NSAID prodrug into nanoparticles, particularly nanoparticles having a hydrophobic core. A nanoparticle having a hydrophobic core can be formed by a hydrophobic polymer or a hydrophobic portion of a polymer, such as a block copolymer. A hydrophobic polymer or hydrophobic portion of a polymer can be a polymer or portion that self-assembles in an aqueous environment.

In various embodiments disclosed herein an aspirin prodrug has the following structure:

(T)ₙ−L−(D)ₘ (I),

where T is a lipophilic moiety; n is a positive integer (such as 1 to 10); D is a releasable NSAID moiety; m is a positive integer (such as 1 to 50); and L is a linker. In some embodiments, T is a saturated or unsaturated, straight or branched chain hydrocarbon having between 4 and 20 carbons. In some embodiments, T is a straight or branched chain C₄-C₂₀ alkyl. In some embodiments, T is octanyl. In some embodiments, n is 1. In some embodiments, D is bound to L via an ester linkage. In some embodiments, m is 2 or more. In some embodiments, m is 4. In some embodiments, L is a 2, 2, bis(methoxy)propionyl moiety. In some embodiments, the releasable NSAID moiety is a releasable aspirin moiety.

In various embodiments disclosed herein an aspirin prodrug has the following structure:

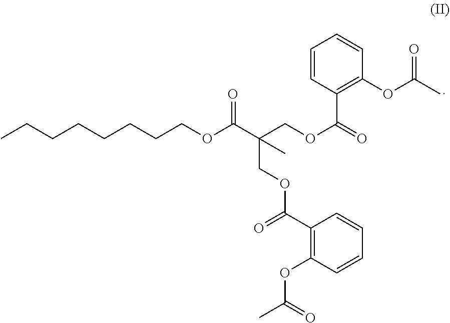 [SMILES: CCCCCCCCOC(=O)C(C)(COC(=O)c1ccccc1OC(C)=O)COC(=O)c1ccccc1OC(C)=O]

In various embodiments disclosed herein an aspirin prodrug has the following structure:

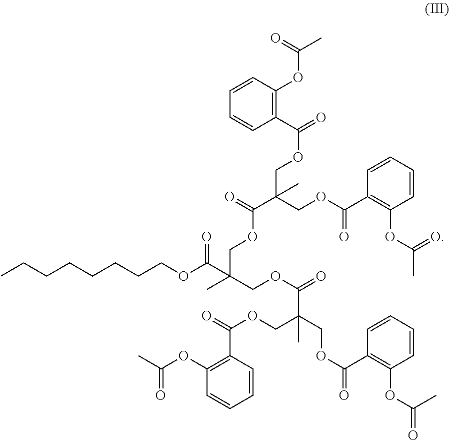 [SMILES: CCCCCCCCOC(=O)C(C)(COC(=O)C(C)(COC(=O)c1ccccc1OC(C)=O)COC(=O)c1ccccc1OC(C)=O)COC(=O)C(C)(COC(=O)c1ccccc1OC(C)=O)COC(=O)c1ccccc1OC(C)=O]

NSAIDs should be released from compounds according to Formulas I, II and III by esterases or acid or base catalyzed reactions in cellular milieu when administered to a subject.

Any suitable NSAID can be modified to form a prodrug as described herein. Examples of suitable NSAIDs include aspirin, salicylates (e.g., sodium, magnesium, choline), celecoxib, diclofenac potassium, diclofenac sodium, diflunisal, etodolac, fenoprofen calcium, flurbiprofen, ibuprofen, indomethacin, ketoprofen, meclofenamate sodium, mefenamic acid, meloxicam, nabumetone, naproxen, naproxen sodium, oxaprozin, piroxicam, rofecoxib, salsalate, sulindac, tolmetin sodium, valdecoxib, and the like. In some preferred embodiments an NSAID modified to form a prodrug as described herein is selected from the group consisting of aspirin (acetyl salicylic acid); salicylic acid; Sulindac Sulfone ((Z)-5-Fluoro-2-methyl-1[p-(methylsulfonyl) benzylidene]indene-3-acetic Acid); Sulindac Sulfide ((Z)-5-Fluoro-2-methyl-1-[p-(methylthio)benzylidene]indene-3-acetic Acid); SC-560 (5-(4-Chlorophenyl)-1-(4-methoxyphenyl)-3-trifluoromethylpyrazole); Resveratrol (trans-3,4,5-Trihydroxystilbene); Pterostilbene succinate, ((E)-4-(4-(3, 5-dimethoxystyryl)phenoxy)-4-oxobutanoic acid); Meloxicam (4-((2-methyl-3-((5-methylthiazol-2-yl)carbamoyl)-1,1-dioxido-2H-benzo[e][1,2]thiazin-4-yl)oxy)-4-oxobutanoic acid); Indomethacin Ester, 4-Methoxyphenyl-(1-(p-Chlorobenzoyl)-5-methoxy-2-methyl-1H-indole-3-acetic Acid, 4-Methoxyphenyl Ester; Indomethacin 1-(p-Chlorobenzoyl)-5-methoxy-2-methyl-1H-indole-3-acetic Acid; Ibuprofen; Flurbiprofen (±)-2-Fluoro-a-methyl[1,1′-biphenyl]-4-acetic Acid); Diclofenac Sodium (2-[(2,6-Dichlorophenyl)amino]benzeneacetic Acid, Sodium); Diclofenac, 4′-Hydroxy-(2-[((2′,6′-Dichloro-4′-hydroxy) phenyl)amino]benzeneacetic Acid) and COX-2 Inhibitor I (Methyl [5-methyl sulfonyl-1-(4-chlorobenzyl)-1H-2-indolyl]carboxylate).

As indicted above, the NSAID prodrugs described herein can be incorporated into nanoparticles. Preferably, the nanoparticle has a hydrophobic core. In some embodiments, the nanoparticle has a mitochondria targeting moiety. In some embodiments, the nanoparticle has a diameter of 150 nanometers or less. Nanoparticles having such diametric dimensions may be better able to cross the blood brain barrier.

Examples of nanoparticles into which a compound according to Formula I, II or III can be incorporated include nanoparticles as described in, for example, Published PCT Patent Application WO 2013/033513, entitled Apoptosis-Targeting Nanoparticles; Published PCT Patent Application WO 2013/123298, entitled Nanoparticles for Mitochondrial Trafficking of Agents; Published PCT Patent Application WO 2014/124425, entitled Generation of Functional Dendritic Cells; and Published PCT Patent Application WO 2014/169007, entitled Combination Therapeutic Nanoparticles, and PCT patent application PCT/US2015/043398, filed on Aug. 3, 2015 and entitled Therapeutic Nanoparticles for Accumulation in the Brain, each of which published patent application is hereby incorporated herein in their respective entireties to the extent that they do not conflict with the disclosure presented herein. A nanoparticle can incorporate one or more targeting moiety, such as a targeting moiety, such as a mitochondria targeting moiety.

I. Core

The core of a nanoparticle may be formed from any suitable component or components. Preferably, the core is formed from hydrophobic components such as hydrophobic polymers or hydrophobic portions of polymers. The core may also or alternatively include block copolymers that have hydrophobic portions and hydrophilic portions that may self-assemble in an aqueous environment into particles having the hydrophobic core and a hydrophilic outer surface. In embodiments, the core comprises one or more biodegradable polymer or a polymer having a biodegradable portion.

Any suitable synthetic or natural bioabsorbable polymers may be used. Such polymers are recognizable and identifiable by one or ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable.

In various embodiments, described herein the core comprises PLGA. PLGA is a well-known and well-studied hydrophobic biodegradable polymer used for the delivery and release of therapeutic agents at desired rates.

Preferably, the at least some of the polymers used to form the core are amphiphilic having hydrophobic portions and hydrophilic portions. The hydrophobic portions can form the core, while the hydrophilic regions may form a layer surrounding the core to help the nanoparticle evade recognition by the immune system and enhance circulation half-life. Examples of amphiphilic polymers include block copolymers having a hydrophobic block and a hydrophilic block. In embodiments, the core is formed from hydrophobic portions of a block copolymer, a hydrophobic polymer, or combinations thereof.

The ratio of hydrophobic polymer to amphiphilic polymer may be varied to vary the size of the nanoparticle. In embodiments, a greater ratio of hydrophobic polymer to amphiphilic polymer results in a nanoparticle having a larger diameter. Any suitable ratio of hydrophobic polymer to amphiphilic polymer may be used. In embodiments, the nanoparticle includes about a 50/50 ratio by weight of amphiphilic polymer to hydrophobic polymer or ratio that includes more amphiphilic polymer than hydrophilic polymer, such as about a 20/80 ratio, about a 30/70 ratio, about a 20/80 ratio, about a 55/45 ratio, about a 60/40 ratio, about a 65/45 ratio, about a 70/30 ratio, about a 75/35 ratio, about a 80/20 ratio, about a 85/15 ratio, about a 90/10 ratio, about a 95/5 ratio, about a 99/1 ratio, or about 100% amphiphilic polymer.

In embodiments, the hydrophobic polymer comprises PLGA, such as PLGA-COOH or PLGA-OH or PLGA-TPP. In embodiments, the amphiphilic polymer comprises PLGA and PEG, such as PLGA-PEG. The amphiphilic polymer may be a dendritic polymer having branched hydrophilic portions. Branched polymers may allow for attachment of more than moiety to terminal ends of the branched hydrophilic polymer tails, as the branched polymers have more than one terminal end.

Nanoparticles having a diameter of about 250 nm or less are generally more effectively targeted to mitochondria than nanoparticles having a diameter of greater than about 250 nm. Nanoparticles having a diameter of about 100 nm or less are generally more effective in crossing the blood-brain barrier. In embodiments, a nanoparticle effective for mitochondrial targeting has a diameter of about 200 nm or less, 190 nm or less, about 180 nm or less, about 170 nm or less, about 160 nm or less, about 150 nm or less, about 140 nm or less, about 130 nm or less, about 120 nm or less, about 110 nm or less, about 100 nm or less, about 90 nm or less, about 80 nm or less, about 80 nm or less, about 80 nm or less, about 80 nm or less, about 80 nm or less, about 70 nm or less, about 60 nm or less, about 50 nm or less, about 40 nm or less, about 30 nm or less, about 20 nm or less, or about 10 nm or less. In embodiments, a nanoparticle has a diameter of from about 10 nm to about 250 nm, such as from about 20 nm to about 200 nm, from about 50 nm to about 160 nm, from about 60 nm to about 150 nm, from about 70 nm to about 130 nm, from about 80 nm to about 120 nm, from about 80 nm to about 100 nm, or the like.

II. Hydrophilic Layer Surrounding the Core

The nanoparticles described herein may optionally include a hydrophilic layer surrounding the hydrophilic core. The hydrophilic layer may assist the nanoparticle in evading recognition by the immune system and may enhance circulation half-life of the nanoparticle.

As indicated above, the hydrophilic layer may be formed, in whole or in part, by a hydrophilic portion of an amphiphilic polymer, such as a block co-polymer having a hydrophobic block and a hydrophilic block.

Any suitable hydrophilic polymer or hydrophilic portion of an amphiphilic polymer may form the hydrophilic layer or portion thereof. The hydrophilic polymer or hydrophilic portion of a polymer may be a linear or dendritic polymer. Examples of suitable hydrophilic polymers include polysaccharides, dextran, chitosan, hyaluronic acid, polyethylene glycol, polymethylene oxide, and the like.

In embodiments, a hydrophilic portion of a block copolymer comprises polyethylene glycol (PEG). In embodiments, a block copolymer comprises a hydrophobic portion comprising PLGA and a hydrophilic portion comprising PEG.

A hydrophilic polymer or hydrophilic portion of a polymer may contain moieties that are charged under physiological conditions, which may be approximated by a buffered saline solution, such as a phosphate or citrate buffered saline solution, at a pH of about 7.4, or the like. Such moieties may contribute to the charge density or zeta potential of the nanoparticle. Zeta potential is a term for electrokinetic potential in colloidal systems. While zeta potential is not directly measurable, it can be experimentally determined using electrophoretic mobility, dynamic electrophoretic mobility, or the like.

It has been found that zeta potential may play an important role in the ability of nanoparticles to accumulate in mitochondria, with higher zeta potentials generally resulting in increased accumulation in the mitochondria. In embodiments, the nanoparticles have a zeta potential, as measured by dynamic light scattering, of about 0 mV or greater. For example, a nanoparticle may have a zeta potential of about 1 mV or greater, of about 5 mV or greater, of about 7 mV or greater, or about 10 mV or greater, or about 15 mV or greater, of about 20 mV or greater, about 25 mV or greater, about 30 mV or greater, about 34 mV or greater, about 35 mV or greater, or the like. In embodiments, a nanoparticle has a zeta potential of from about 0 mV to about 100 mV, such as from about 1 mV to 50 mV, from about 2 mV to about 40 mV, from about 7 mV to about 35 mV, or the like.

Any suitable moiety that may be charged under physiological conditions may be a part of or attached to a hydrophilic polymer or hydrophilic portion of a polymer. In some embodiments, the moiety is present at a terminal end of the polymer or hydrophilic portion of the polymer. Of course, the moiety may be directly or indirectly bound to the polymer backbone at a location other than at a terminal end. Due to the substantial negative electrochemical potential maintained across the inner mitochondrial membrane, cations, particularly if delocalized, are effective at crossing the hydrophobic membranes and accumulating in the mitochondrial matrix. Cationic moieties that are known to facilitate mitochondrial targeting are discussed in more detail below. However, cationic moieties that are not particularly effective for selective mitochondrial targeting may be included in nanoparticles or be bound to hydrophilic polymers or portions of polymers. In embodiments, anionic moieties may form a part of or be attached to the hydrophilic polymer or portion of a polymer. The anionic moieties or polymers containing the anionic moieties may be included in nanoparticles to tune the zeta potential, as desired. In embodiments, a hydrophilic polymer or portion of a polymer includes a hydroxyl group that can result in an oxygen anion when placed in a physiological aqueous environment. In embodiments, the polymer comprises PEG-OH where the OH serves as the charged moiety under physiological conditions.

III. Mitochondria Targeting Moieties

The nanoparticles described herein include one or more moieties that target the nanoparticles to mitochondria. As used herein, “targeting” a nanoparticle to mitochondria means that the nanoparticle accumulates in mitochondria relative to other organelles or cytoplasm at a greater concentration than substantially similar non-targeted nanoparticle. A substantially similar non-target nanoparticle includes the same components in substantially the same relative concentration (e.g., within about 5%) as the targeted nanoparticle, but lacks a targeting moiety.

The mitochondrial targeting moieties may be tethered to the core in any suitable manner, such as binding to a molecule that forms part of the core or to a molecule that is bound to the core. In embodiments, a targeting moiety is bound to a hydrophilic polymer that is bound to a hydrophobic polymer that forms part of the core. In embodiments, a targeting moiety is bound to a hydrophilic portion of a block copolymer having a hydrophobic block that forms part of the core.

The targeting moieties may be bound to any suitable portion of a polymer. In embodiments, the targeting moieties are attached to a terminal end of a polymer. In embodiments, the targeting moieties are bound to the backbone of the polymer, or a molecule attached to the backbone, at a location other than a terminal end of the polymer. More than one targeting moiety may be bound to a given polymer. In embodiments, the polymer is a dendritic polymer having multiple terminal ends and the targeting moieties may be bound to more than one of terminal ends.

The polymers, or portions thereof, to which the targeting moieties are bound may contain, or be modified to contain, appropriate functional groups, such as —OH, —COOH, —NH₂, —SH, —N₃, —Br, —Cl, —I, or the like, for reaction with and binding to the targeting moieties that have, or are modified to have, suitable functional groups.

Examples of targeting moieties tethered to polymers presented throughout this disclosure for purpose of illustrating the types of reactions and tethering that may occur. However, one of skill in the art will understand that tethering of targeting moieties to polymers may be carried out according to any of a number of known chemical reaction processes.

Targeting moieties may be present in the nanoparticles at any suitable concentration. In embodiments, the concentration may readily be varied based on initial in vitro analysis to optimize prior to in vivo study or use. In embodiments, the targeting moieties will have surface coverage of from about 5% to about 100%.

Any suitable moiety for facilitating accumulation of the nanoparticle within the mitochondrial matrix may be employed. Due to the substantial negative electrochemical potential maintained across the inner mitochondrial membrane, delocalized lipophilic cations are effective at crossing the hydrophobic membranes and accumulating in the mitochondrial matrix. Triphenyl phosophonium (TPP) containing compounds can accumulate greater than 1000 fold within the mitochondrial matrix. Any suitable TPP-containing compound may be used as a mitochondrial matrix targeting moiety. Representative examples of TPP-based moieties may have structures indicated below in Formula IV, Formula V or Formula VI:

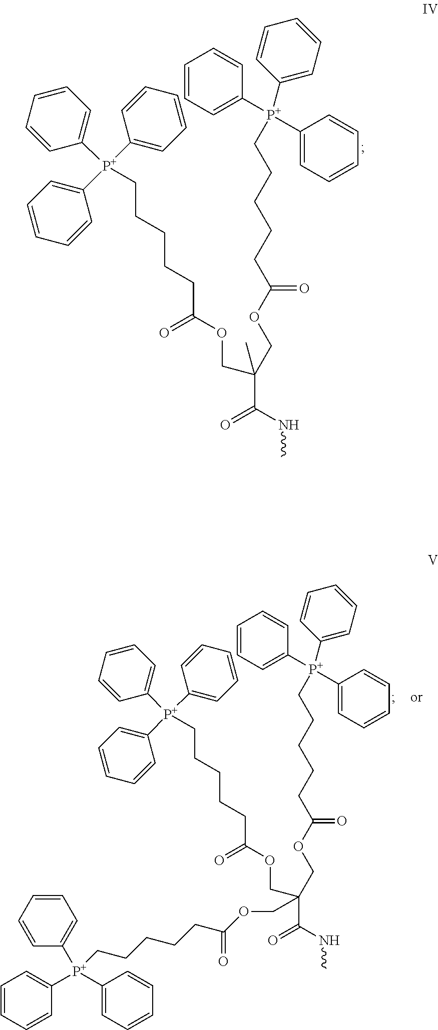

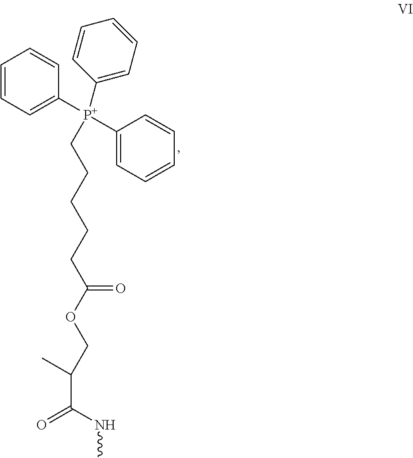

where the amine (as depicted) may be conjugated to a polymer or other component for incorporation into the nanoparticle.

In embodiments, the delocalized lipophilic cation for targeting the mitochondrial matrix is a rhodamine cation, such as Rhodamine 123 having Formula VII as depicted below:

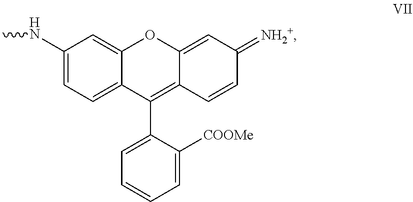

where the secondary amine (as depicted) may be conjugated to a polymer, lipid, or the like for incorporation into the nanoparticle.

Of course, non-cationic compounds may serve to target and accumulate in the mitochondrial matrix. By way of example, Szeto-Shiller peptide may serve to target and accumulate a nanoparticle in the mitochondrial matrix. Any suitable Szetto-Shiller peptide may be employed as a mitochondrial matrix targeting moiety. Non-limiting examples of suitable Szeto-Shiller peptides include SS-02 and SS-31, having Formula VIII and Formula IX, respectively, as depicted below:

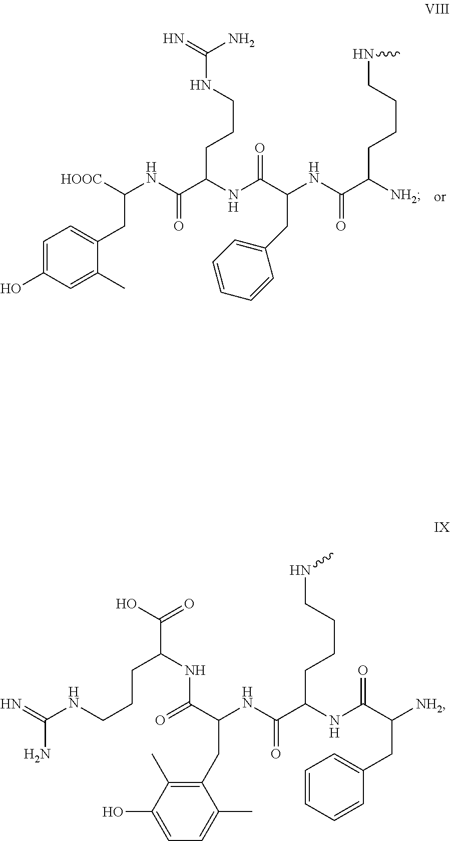

where the secondary amine (as depicted) may be conjugated to a polymer, lipid, or the like for incorporation into the nanoparticle.

For purposes of example, a reaction scheme for synthesis of PLGA-PEG-TPP is shown below in Scheme I. It will be understood that other schemes may be employed to synthesize PLGA-PEG-TPP and that similar reaction schemes may be employed to tether other mitochondrial targeting moieties to PLGA-PEG or to tether moieties to other polymer or components of a nanoparticle.

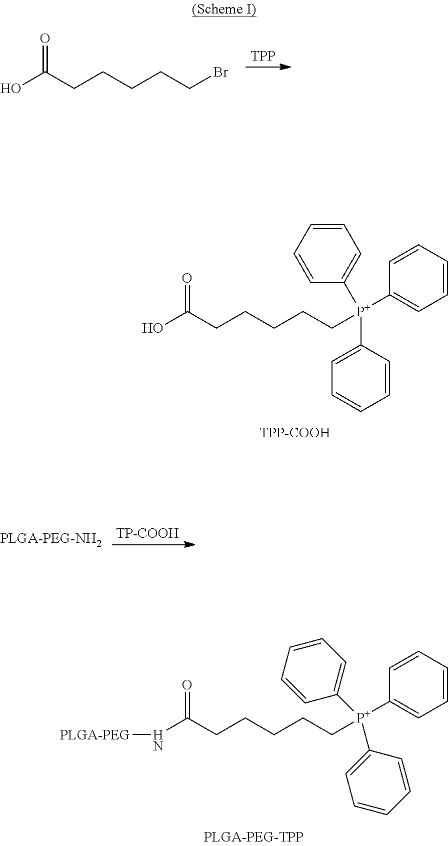

Preferably, a targeting moiety is attached to a hydrophilic polymer or hydrophilic portion of a polymer so that the targeting moiety will extend from the core of the nanoparticle to facilitate the effect of the targeting moiety.

It will be understood that the mitochondrial targeting moiety may alter the zeta potential of a nanoparticle. Accordingly, the zeta potential of a nanoparticle may be tuned by adjusting the amount of targeting moiety included in the nanoparticle. The zeta potential may also be adjusted by including other charged moieties, such as charged moieties of, or attached to, hydrophilic polymers or hydrophilic portions of polymers.

In embodiments, charged moieties are provided only by, or substantially by, mitochondrial targeting moieties. In embodiments, about 95% or more of the charged moieties are provided by mitochondrial targeting moieties. In embodiments, about 90% or more of the charged moieties are provided by mitochondrial targeting moieties. In embodiments, about 85% or more of the charged moieties are provided by mitochondrial targeting moieties. In embodiments, about 80% or more of the charged moieties are provided by mitochondrial targeting moieties. In embodiments, about 75% or more of the charged moieties are provided by mitochondrial targeting moieties. In embodiments, about 70% or more of the charged moieties are provided by mitochondrial targeting moieties. In embodiments, about 65% or more of the charged moieties are provided by mitochondrial targeting moieties. In embodiments, about 60% or more of the charged moieties are provided by mitochondrial targeting moieties. In embodiments, about 55% or more of the charged moieties are provided by mitochondrial targeting moieties. In embodiments, about 50% or more of the charged moieties are provided by mitochondrial targeting moieties. Of course, the mitochondrial targeting moieties may provide any suitable amount or percentage of the charged moieties.

In embodiments, the nanoparticles are formed by blending a polymer that include a mitochondrial targeting moiety with a polymer that includes a charged moiety other than a mitochondrial targeting moiety.

IV. Cell Penetration or Brain Accumulation Moieties

A nanoparticle as described herein can include any suitable moiety to enhance penetration of the nanoparticle into a cell or to accumulate the nanoparticle in the brain. Any known cell penetration enhancer or brain accumulation moiety can be used and can be bound to, for example, a polymer for incorporation into the nanoparticle. The moieties can be attached to a polymer in a similar manner to the mitochondria targeting moieties as described herein or in any other suitable manner.

Examples of cell penetrating moieties include cell penetrating peptides (CPPs), which are short peptides that facilitate cellular uptake, and the like.

Examples of brain accumulation moieties include moieties that bind to receptors, cell adhesion proteins, or other available molecules selectively presented on cells in the brain, moieties that exploit trafficking properties of the blood-brain barrier, or the like. For example, suitable brain accumulation moieties include cationic proteins or CPPS that trigger electrostatic interaction between positively charged moieties of the proteins and negatively charged membrane surface regions on the brain endothelial cells. Cationic serum albumin is one example. Other examples include proteins or peptides of transcription-activating factor Tat, penetratin, and the Syn-B vectors. Other examples include moieties that take advantage of glucose transporter to cross the blood-brain barriers. Mannose is one example. Yet other examples include moieties that take advantage of choline transporters, such as quaternary ammonium ligands. Still other examples include those that bind to transferrin receptors, low density lipoprotein receptors, insulin receptors, nicotinic acetylcholine receptors or other receptors selectively present on the capillary endothelium of the brain.

In some embodiments, a transferrin moiety is coupled to a polymer for incorporation into a nanoparticle. In some embodiments, an aprotinin or angiopep moiety is coupled to a polymer for incorporation into a nanoparticle. In some embodiments, a CDX peptide or other nicotinic acetylcholine receptor binding moiety is coupled to a polymer for incorporation into a nanoparticle.

V. Synthesis of Nanoparticle

Nanoparticles, as described herein, may be synthesized or assembled via any suitable process.

Preferably, the nanoparticles are assembled in a single step to minimize process variation. A single step process may include nanoprecipitation and self-assembly.

In general, the nanoparticles may be synthesized or assembled by dissolving or suspending hydrophobic components in an organic solvent, preferably a solvent that is miscible in an aqueous solvent used for precipitation. In embodiments, acetonitrile is used as the organic solvent, but any suitable solvent (such as DMF, DMSO, acetone, or the like) may be used. Hydrophilic components are dissolved in a suitable aqueous solvent, such as water, 4 wt-% ethanol, or the like. The organic phase solution may be added drop wise to the aqueous phase solution to nanoprecipitate the hydrophobic components and allow self-assembly of the nanoparticle in the aqueous solvent.

A process for determining appropriate conditions for forming the nanoparticles may be as follows. Briefly, functionalized polymers and other components, if included or as appropriate, may be co-dissolved in organic solvent mixtures. This solution may be added drop wise into hot (e.g, 65° C.) aqueous solvent (e.g, water, 4 wt-% ethanol, etc.), whereupon the solvents will evaporate, producing nanoparticles with a hydrophobic core surrounded by a hydrophilic polymer component, such as PEG. Once a set of conditions where a high (e.g., >75%) level of targeting moiety surface loading has been achieved, contrast agents or therapeutic agents may be included in the nanoprecipitation and self-assembly of the nanoparticles.

If results are not desirably reproducible by manual mixing, microfluidic channels may be used.

Nanoparticles may be characterized for their size, charge, stability, IO and QD loading, drug loading, drug release kinetics, surface morphology, and stability using well-known or published methods.

Nanoparticle properties may be controlled by (a) controlling the composition of the polymer solution, and (b) controlling mixing conditions such as mixing time, temperature, and ratio of water to organic solvent. The likelihood of variation in nanoparticle properties increases with the number of processing steps required for synthesis.

The size of the nanoparticle produced can be varied by altering the ratio of hydrophobic core components to amphiphilic shell components. Nanoparticle size can also be controlled by changing the polymer length, by changing the mixing time, and by adjusting the ratio of organic to the phase. Prior experience with nanoparticles from PLGA-b-PEG of different lengths suggests that nanoparticle size will increase from a minimum of about 20 nm for short polymers (e.g. PLGA₃₀₀₀-PEG₇₅₀) to a maximum of about 150 nm for long polymers (e.g. PLGA_{100,000}-PEG_{10,000}). Thus, molecular weight of the polymer will serve to adjust the size.

Nanoparticle surface charge can be controlled by mixing polymers with appropriately charged end groups. Additionally, the composition and surface chemistry can be controlled by mixing polymers with different hydrophilic polymer lengths, branched hydrophilic polymers, or by adding hydrophobic polymers.

Once formed, the nanoparticles may be collected and washed via centrifugation, centrifugal ultrafiltration, or the like. If aggregation occurs, nanoparticles can be purified by dialysis, can be purified by longer centrifugation at slower speeds, can be purified with the use surfactant, or the like.

Once collected, any remaining solvent may be removed and the particles may be dried, which should aid in minimizing any premature breakdown or release of components. The nanoparticles may be freeze dried with the use of bulking agents such as mannitol, or otherwise prepared for storage prior to use.

It will be understood that therapeutic agents may be placed in the organic phase or aqueous phase according to their solubility.

Nanoparticles described herein may include any other suitable components, such as phospholipids or cholesterol components, generally know or understood in the art as being suitable for inclusion in nanoparticles.

VI. Use and Testing

The performance and characteristics of nanoparticles produced herein may be tested or studied in any suitable manner. By way of example, therapeutic efficacy can be evaluated using cell-based assays. Toxicity, bio-distribution, pharmacokinetics, and efficacy studies can be tested in cells or rodents or other mammals. Zebrafish or other animal models may be employed for combined imaging and therapy studies. Rodents, rabbits, pigs, or the like may be used to evaluate diagnostic or therapeutic potential of nanoparticles. Some additional details of studies that may be performed to evaluate the performance or characteristics of the nanoparticles, which may be used for purposes of optimizing the properties of the nanoparticles are described below. However, one of skill in the art will understand that other assays and procedures may be readily performed.

Uptake and binding characteristics of nanoparticles containing a contrast agent may be evaluated in any suitable cell line, such as RAW 264.7, J774, jurkat, and HUVEGs cells. The immunomodulatory role of nanoparticles may be assayed by determining the release of cytokines when these cells are exposed to varying concentrations of nanoparticles. Complement activation may be studied to identify which pathways are triggered using columns to isolate opsonized nanoparticles; e.g. as described in Salvador-Morales C, Zhang L, Langer R, Farokhzad O C, Immunocompatibility properties of lipid-polymer hybrid nanoparticles with heterogeneous surface functional groups, Biomaterials 30: 2231-2240, (2009). Fluorescence measurements may be carried out using a plate reader, FACS, or the like. Because nanoparticle size is an important factor that determines biodistribution, Nanoparticles may be binned into various sizes (e.g., 20-40, 40-60, 60-80, 80-100, 100-150, and 150-300 nm) and tested according to size.

Biodistribution (bioD) and pharmacokinetic (PK) studies may be carried out in rats or other suitable mammals. For PK and bioD analysis, Sprague Dawley rats may be dosed with QD-labeled, apoptosis-targeting, macrophage-targeting nanoparticles or similar nanoparticles without the targeting groups, through a lateral tail vein injection. The bioD may be followed initially by fluorescence imaging for 1-24 h after injection. Animals may be sacrificed; and brain, heart, intestine, liver, spleen, kidney, muscle, bone, lung, lymph nodes, gut, and skin may be excised, weighed, homogenized, and Cd from QD may be quantified using ICP-MS. Tissue concentration may be expressed as % of injected dose per gram of tissue (% ID/g). Blood half-life may be calculated from blood Cd concentrations at various time points

Therapeutic dosages of nanoparticles effective for human use can be estimated from animal studies according to well-known techniques, such as surface area or weight based scaling.

The nanoparticles described herein can be administered to patients in need thereof. Because the nanoparticles include an aspirin prodrug, the nanoparticles can be used to inhibit cyclooxygenase. In some embodiments, the nanoparticles can be used to treat an inflammatory disease. In some embodiments the nanoparticles can be used to treat a neurodegenerative disease.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise. The use of “and/or” in certain locations is not intended mean that the use of “or” in other locations cannot mean “and/or.”

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “disease” means a condition of a living being or one or more of its parts that impairs normal functioning. As used herein, the term disease encompasses terms such disease, disorder, condition, dysfunction and the like.

As used herein, “treat” or the like means to cure, prevent, or ameliorate one or more symptom of a disease.

As used herein, “bind,” “bound,” “conjugated” or the like means that chemical entities are joined by any suitable type of bond, such as a covalent bond, an ionic bond, a hydrogen bond, van der walls forces, or the like. “Bind,” “bound,” and the like are used interchangeable herein with “attach,” “attached,” and the like. Preferably, “conjugated” is used herein to refer to a covalent bond.

A compound as described herein may contain one or more chiral centers and/or double bonds and, therefore, exist as stereoisomers, such as double-bond isomers (i.e., geometric isomers), enantiomers, or diastereomers. For purposes of the present disclosure, chemical structures depicted herein, including a compound according to Formula I, encompass all of the corresponding compounds' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds of this invention are preferred.

When a disclosed compound is named or depicted by structure, it is to be understood that solvates (e.g., hydrates) of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include a stoichiometric or non-stoichiometric amount of water bound by non-covalent intermolecular forces.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e. the capacity to occur in different crystalline forms). These different crystalline forms are typically known as “polymorphs.” It is to be understood that when named or depicted by structure, the disclosed compounds and solvates (e.g., hydrates) also include all polymorphs thereof. As used herein, the term “polymorph” means solid crystalline forms of a compound or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salts, solvates or polymorphs are also included. As used herein, the term “clathrate” means a compound of the present invention or a salt thereof in the form of a crystal lattice that contains spaces (e.g., channels) that have a guest molecule (e.g., a solvent or water) trapped within.

Provided below are non-limiting examples of specific embodiments, of compounds, nanoparticles and methods described herein.

### EXAMPLES

For better use of cyclooxygenase dependent anti-inflammatory properties and mitochondrial activities of aspirin for its possible use as a neuroprotectant, new hydrophobic analogues of aspirin were developed and successfully encapsulated in polymeric nanoparticles. Anti-inflammatory effects of these NPs in vivo using a mouse model demonstrated unique properties of the new hydrophobic aspirin analogue to inhibit production of pro-inflammatory and enrichment of anti-inflammatory cytokines.

Conditions that include neuro-inflammation, oxidative stress, and mitochondrial injury play different roles in the prognosis of neurodegenerative diseases such as stroke, Alzheimer's disease, Parkinson's disease (PD), Huntington's disease, and amyotrophic lateral sclerosis. Although these diseases demonstrate different pathologies, inflammation and oxidative stress are the common players. Degradation in mitochondrial health also plays an integral role in overall damage during neuro-degeneration. Anti-inflammatory substances such as aspirin and mitochondria-acting antioxidant coenzyme Q₁₀ are described to have potential neuroprotective roles in these diseases. Aspirin or acetylsalicylic acid can potentially have a number of roles in neurodegenerative diseases: (i) platelet inhibition through acetylation and prevention of new clots from developing, (ii) aspirin can play roles in PD by suppressing formation of dopamine quinone, (iii) cyclooxygenase (COX)-independent effect of aspirin on Ca²⁺ signaling for mitochondrial dysfunction related neuro-degeneration.

Current knowledge and clinical data indicate that aspirin can be an attractive addition to treatment regiments for neurodegenerative diseases. By acknowledging the fact that although few of the nonsteroidal anti-inflammatory drugs such as aspirin can get access to the brain tissue by crossing the tight junctions of the blood brain barrier (BBB), but plasma protein binding activity of this class of molecule limits the effectiveness of such uptake, we hypothesized that new hydrophobic analogues of aspirin can be extremely important as aspirin lacks properties required for well formulation in a nanoparticle (NP) system for better delivery. Furthermore, gastric toxicity arising from non-specific platelet inhibition by aspirin is a major problem and one of the solutions can be slow-release of aspirin at low dosage. Thus NPs with controlled release properties can provide beneficial manipulation towards pharmacological formulation for aspirin. Additionally, hydrophobic aspirin analogues will help in improving pharmacokinetic (PK) parameters of the generic drug as incorporation of new derivatives into a NP system can increase the blood circulation time of the drug when administered by intravenous (i.v.) route in contrast to usual aspirin administration.

Here we report construction and optimization of new aspirin analogues for their formulation in biodegradable NPs with properties which will allow slow controlled delivery of aspirin molecules in the vicinity of the target tissue and in particular in the mitochondria for possible applications in neuro-degenerative diseases. As a disease model, we investigated utilities of these new aspirin-NP formulations in mice model of inflammation. Prior to the synthesis of new aspirin derivatives, we first assessed whether generic aspirin can be incorporated in the hydrophobic core of the biodegradable polymeric NPs. As we would like to target conditions such as mitochondrial dysfunctions associated with oxidative stress, impaired Ca²⁺ signaling, neuro-inflammatory processes demonstrated by brain cells during neurodegenerative processes, we selected a biodegradable poly(lactic-co-glycolic acid)-block-polyethyleneglycol (PLGA-b-PEG) functionalized with a terminal triphenylphosphonium cation (TPP) with significant mitochondrial association properties previously reported by Marrache and Dhar, Proc. Natl. Acad. Sci. USA, 2012, 109:16288-16293. In addition, recent studies demonstrated that well-optimized NPs from this polymer show brain accumulation properties. See, e.g., Marrache et al., Proc. Natl. Acad. Sci. USA, 2014, 111:10444-10449; and Feldhaeusser et al., Nanoscale, 2015, 7:13822-13830.

In our continuing effort to evaluate the potential of the targeted NPs (T-NPs) derived from this PLGA-b-PEG-TPP polymer to deliver payload that can work by accessing unique targets at the mitochondria, we first evaluated whether aspirin (Asp) can be incorporated in the T-NPs. Nanoprecipitation of non-targeted PLGA-b-PEG-OH polymer or targeted PLGA-b-PEG-TPP polymer in presence of aspirin afforded low encapsulation efficiency (EE) and percent loading of aspirin inside NT/T-Asp-NPs. Poor encapsulation of aspirin inside the hydrophobic core arises from hydrophilic properties of aspirin. Thus, we hypothesized that construction of hydrophobic analogues which can release aspirin by taking advantages of the hydrolytic agents present in the cellular milieu can be attractive strategy for better delivery of aspirin at the target with improved PK and biodistribution (bioD) properties when administered in vivo.

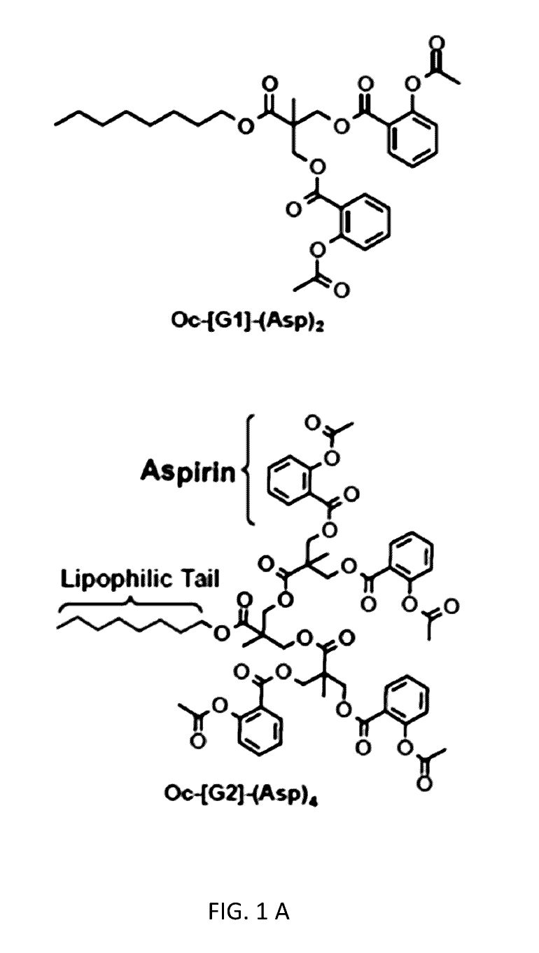

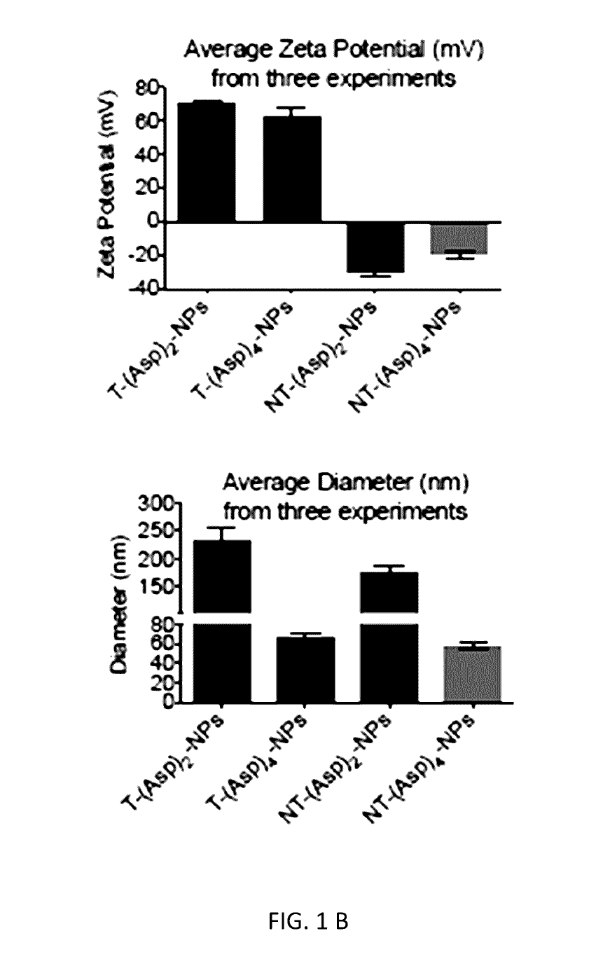

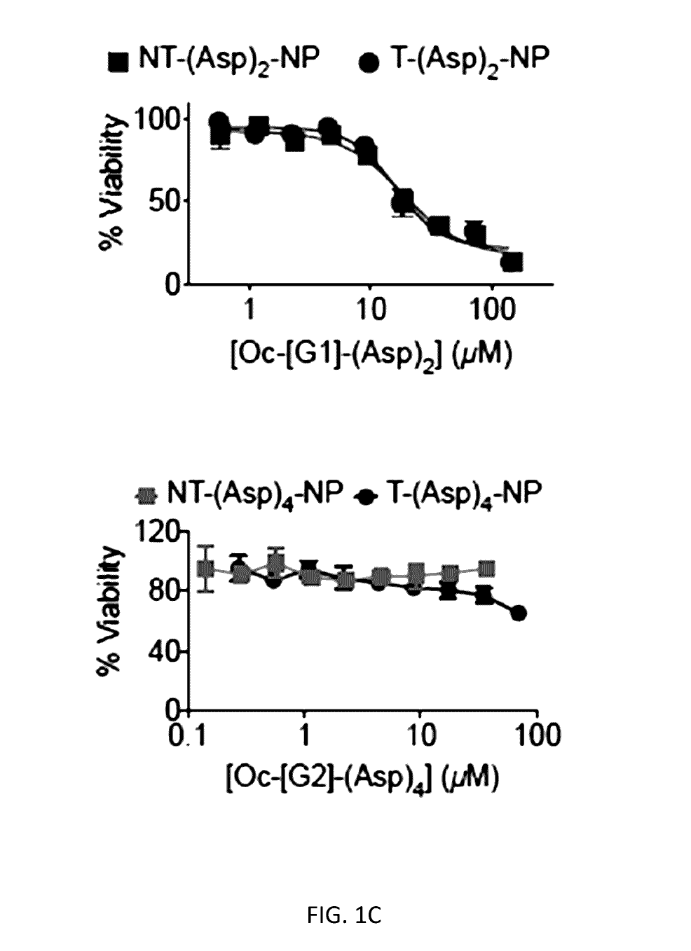

Analyses of the properties for incorporation of aspirin inside hydrophobic core and to increase therapeutic efficacy prompted us to explore the possibility of use of a hydrophobic dendritic platform as the number of aspirin moieties can easily be tuned. We first developed a first generation [G1] hydrophobic biodegradable dendron with an octyl (Oc) chain connected to two available —OH moieties Oc-[G1]-(OH)₂ (4) for conjugation of two aspirin molecules. This dendron Oc-[G1]-(OH)₂ was reacted with aspirin chloride to generate a hydrophobic dendron Oc-[G1]-(Asp)₂ containing two molecules of aspirin linked through cleavable ester bonds (FIG. 1A). Our efforts to encapsulate Oc-[G1]-(Asp)₂ in PLGA-b-PEG-TPP polymer to generate T-(Asp)₂-NPs and in PLGA-b-PEG-OH polymer to yield NT-(Asp)₂-NPs resulted in high loading of the dendron inside the NPs, however the diameter of both T/NT-(Asp)₂-NPs were ˜200 nm (FIG. 1B) which may disqualify these NPs to be suitable for either brain accumulation or mitochondrial association as previous studies indicated that NP size below 100 nm is desired for both of these properties. See, e.g., Marrache and Dhar, Proc. Natl. Acad. Sci. USA, 2012, 109:16288-16293; Marrache et al., Proc. Natl. Acad. Sci. USA, 2014, 111:10444-10449; and Feldhaeusser et al., Nanoscale, 2015, 7:13822-13830.

Next, we increased the number of arms of dendron further to increase hydrophobicity of the Dendron and constructed Oc-[G2]-(OH)₄ and further conjugation of aspirin resulted in Oc-[G2]-(Asp)₄ (FIG. 1A) with four aspirin molecules. Incorporation of Oc-[G2]-(Asp)₄ into NPs to generate T-(Asp)₄-NP and NT-(Asp)₄-NPs indicated sizes below 100 nm and highly positive surface for the T-NPs (FIG. 1B). Comparison of NP sizes from these two dendrons indicated that Oc-[G2]-(Asp)₄ will be a more appropriate derivative for aspirin delivery. Further, cytotoxicity of T/NT-(Asp)₂-NPs and T/NT-(Asp)₄-NPs in RAW 264.7 macrophages indicated that the NPs derived from Oc-[G1]-(Asp)₂ are relatively more toxic to the cells whereas the NPs from Oc-[G2]-(Asp)₄ did not demonstrate any such toxicity up to 100 μM (with respect to aspirin present in the NPs) (FIG. 1C). Based on the size of the NPs and toxicity, we decided to use Oc-[G2]-(Asp)₄ for delivery of aspirin using NP platform.

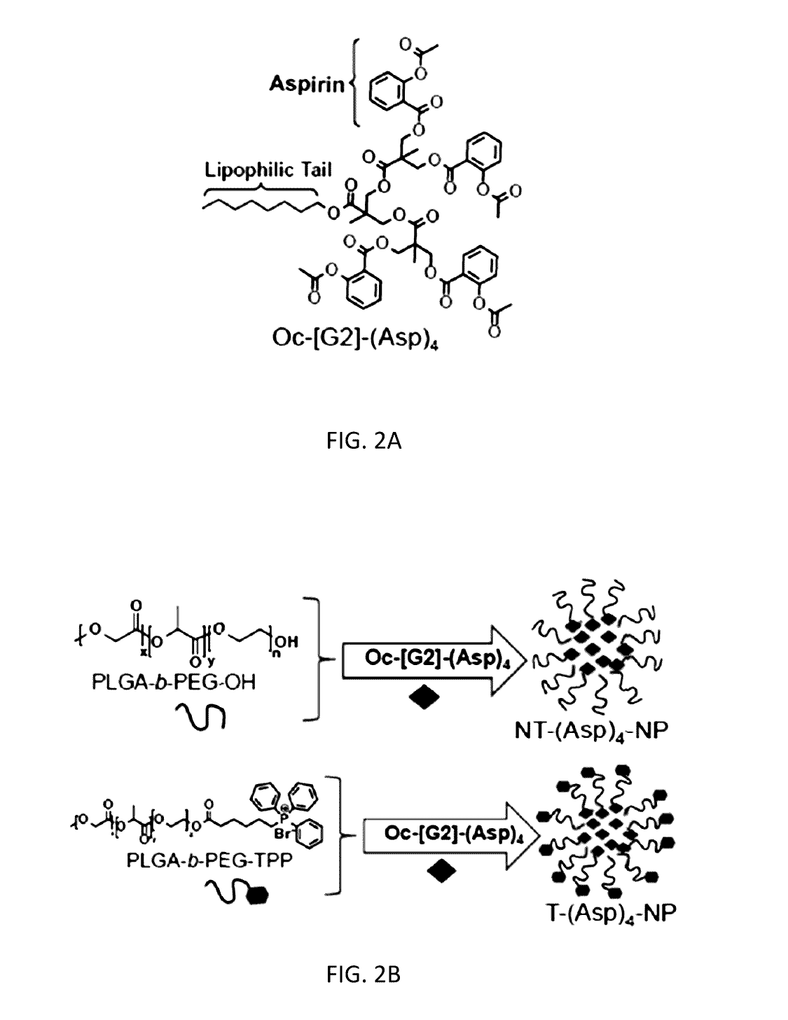

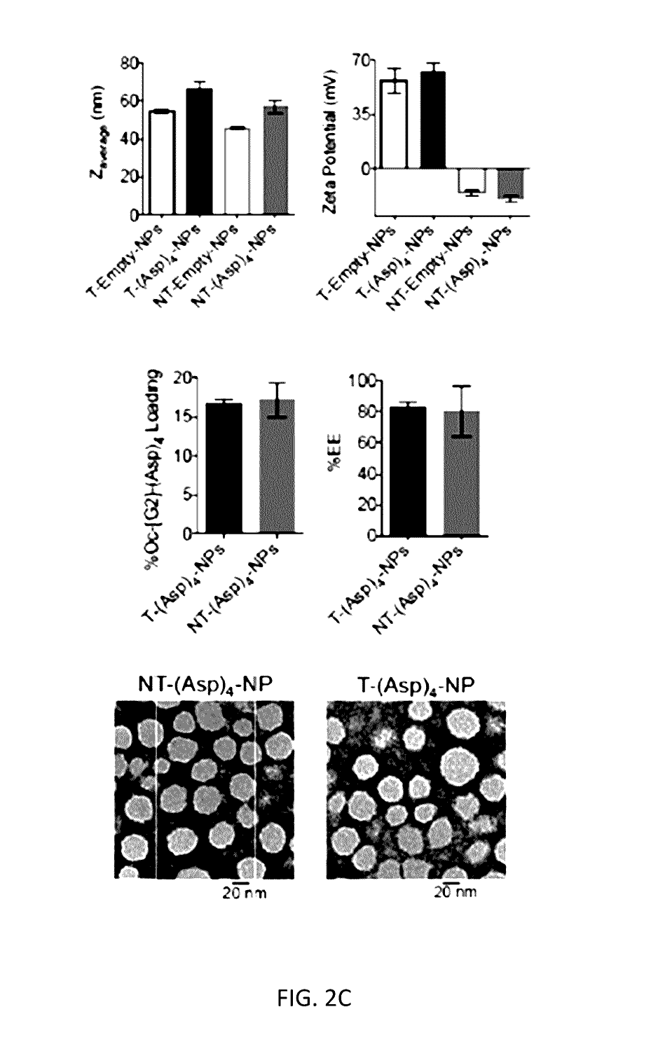

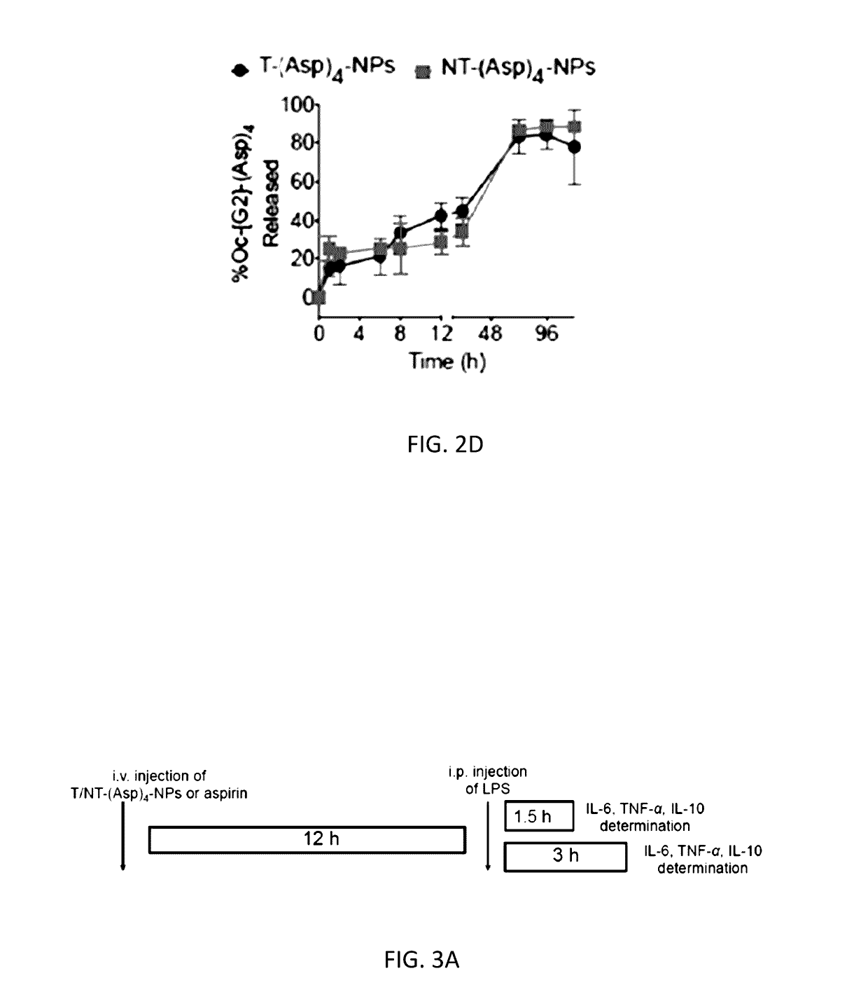

Nanoprecipitation was carried out using 20% feed of Oc-[G2]-(Asp)₄ (FIG. 2A) with PLGA-b-PEG-TPP polymer to result in T-(Asp)₄-NPs or with PLGA-b-PEG-OH polymer to produce NT-(Asp)₄-NPs (FIG. 2B). Control empty-T/NT-NPs were also prepared. Dynamic light scattering (DLS) studies indicated that these NPs have diameter below 100 nm; T-NPs demonstrated high positively charged surface, and the NT-NPs were negatively charged (FIG. 2C). Determination of percent Oc-[G2]-(Asp)₄ loading by high performance liquid chromatography (HPLC) indicated high loadings of 17±2% for NT and 16.6±0.6% for T NPs, respectively (FIG. 2C). Transmission electron microscopy (TEM) based analyses of the NPs further supported the diameter and confirmed that these spherical NPs are homogeneous (FIG. 2C). Studies suggest that aspirin is an antiplatelet agent that can be effective as an early treatment in acute ischemic stroke and aspirin therapy should be used within 48 h of the initiation of symptoms. This made us realize that although controlled release NPs can be invaluable addition to aspirin therapeutic regiments, but the NPs should have release properties where significant portion of aspirin can get released in ˜48 h. Investigation of release kinetics of aspirin derivative from T/NT-(Asp)₄-NPs under physiological conditions of pH 7.4 at 37° C. demonstrated release of ˜50% Oc-[G2]-(Asp)₄ indicating that these NPs are suitable for aspirin delivery for neuroprotection (FIG. 2D).

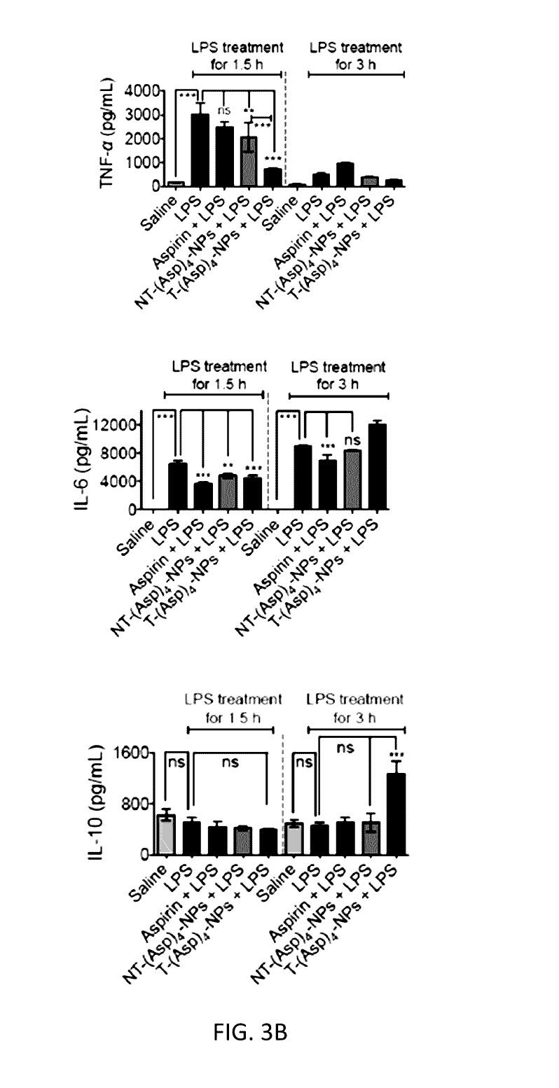

To explore the anti-inflammatory properties of the new aspirin derivative in NP formulation in vivo, we used mice stimulated with lipopolysaccharide (LPS). An earlier study demonstrated that intraperitonially injected LPS can cause secretion of significant amounts of TNF-α, which peaks around at 1.5 h and IL-6 at around 3 h after administration. In our studies following similar protocol in C57BL/6 or BALB/c (albino) mice, we observed enhanced levels of TNF-α and IL-6 in the serum after intraperitonial administration of 100 μg of LPS per animal and the levels peaked at 1.5 and 3 h, respectively for TNF-α and IL-6. Next, 8 week old BALB/c male mice were administered with saline or aspirin (20 mg/kg) or NT-(Asp)₄-NP (20 mg/kg with respect to aspirin) or T-(Asp)₄-NP (20 mg/kg with respect to aspirin) by i.v. injections and after 12 h, these animals were subsequently treated with intraperitonially injected LPS for 1.5 h and 3 h (FIG. 3A). Serum samples were isolated from the treated animals and pro-inflammatory and anti-inflammatory cytokine levels in the serum samples were evaluated by the enzyme-linked immunosorbent assay (ELISA). As seen with C57BL/6 mice, serum TNF-α and IL-6 levels were increased after administration of LPS in BALB/c Albino mice following similar patterns where TNF-α level was peaked at ˜1.5 h and maximum IL-6 level was found at −3 h and these levels were significantly higher (P<0.001) than only saline treated groups (FIG. 3B). Preventative treatment with aspirin (20 mg/kg) followed by LPS for 1.5 h, TNF-α level was less than LPS alone, but these differences did not reach any statistical significance (LPS vs. aspirin+LPS: non-significant for TNF-α, FIG. 3B). Serum samples from the group of animals treated with NT-(Asp)₄-NPs prior to LPS treatment for 1.5 h had significantly lower TNF-α than only LPS treated group (P=0.001-0.01). Significantly, TNF-α levels from the animals treated with T-(Asp)₄-NPs followed by LPS treatment for 1.5 h was drastically reduced compared to only LPS (P<0.001) (FIG. 3B). Serum TNF-α levels in the T-(Asp)₄-NP treated LPS stimulated group was significantly lower than the levels found in the NT-(Asp)₄-NP plus LPS treated animals when 1.5 h time point was considered (P<0.001) (FIG. 3B). Thus, these results indicated that T-(Asp)₄-NPs are considerably more effective than aspirin or NT-(Asp)₄-NPs in inhibiting TNF-α production upon LPS stimulation in vivo. Preventative treatment with aspirin, NT-(Asp)4-NPs, or T-(Asp)4-NPs prior to stimulation with LPS for 3 h did not show any significant differences in serum TNF-α levels as this cytokine declined by 3 h (FIG. 3B). In our experimental conditions, the level of IL-6 in only LPS treated samples was significantly increased from that in saline treated group at 1.5 h and the level increased further when LPS treatment was carried out for 3 h. When LPS treatment for 1.5 h was considered, the IL-6 levels were significantly reduced for the groups where preventative treatments were carried out with aspirin, NT-(Asp)₄-NPs, or T-(Asp)₄-NPs (FIG. 3B). The IL-6 level in the T-(Asp)₄-NP treated group was lower than the group administered with NT-(Asp)₄-NPs at 1.5 h, however the differences between these two groups did not reach statistical significance (FIG. 3B). These observations indicated that the targeted NP formulation of Oc-[G2]-(Asp)₄ is as effective as aspirin in preventing LPS induced IL-6 secretion in vivo. When LPS treatment was carried out for 3 h, only aspirin showed reduced IL-6 levels compared to LPS alone.

Anti-inflammatory IL-10 determination in the serum samples demonstrated no significant amounts of this cytokine at the 1.5 h LPS treated samples. However, when 3 h LPS treatment period was considered, a significantly higher level of this anti-inflammatory cytokine was detected in the serum samples from the animals which were pretreated with T-(Asp)4-NPs prior to LPS stimulation, no other treated group showed such a high IL-10 level (FIG. 3B). The compelling properties of T-(Asp)₄-NPs in inhibiting production of pro-inflammatory cytokines and induction of anti-inflammatory IL-10 indicated that the new formulation of aspirin can be an attractive candidate for further exploration for potential activities in neuro-inflammation.

This work provides first hydrophobic, non-toxic aspirin analogue of aspirin which can be loaded inside polymeric NPs efficiency, thus overcoming the disadvantages arising from physicochemical properties of aspirin which do not allow its encapsulation inside the hydrophobic core of NPs. Conjointly, our findings highlighted potential abilities of this new hydrophobic aspirin analogue Oc-[G2]-(Asp)₄ encapsulated mitochondria targeted NP as a possible therapeutic intervention of the central nervous system inflammation leading to protection against neurodegenerative diseases with inflammatory symptoms. C56BL/6 (12 weeks old) and BALB/c Albino male mice (8 weeks old) were obtained from Charles River Laboratories and handled in accordance with Animal Welfare Act (AWA), and other applicable federal and state guidelines. All animal work presented here was approved by Institutional Animal Care and Use Committee (IACUC) of University of Georgia. All statistical analyses were performed using GraphPad Prism software performing a one-way analysis of variance (ANOVA) and nonparametric analyses followed by the Tukey post-test.

Materials and Methods

Materials:

All chemicals were used as received without further purification unless otherwise noted. Acetylsalisylic acid (aspirin), 2,2 bis(methoxy)propionic acid (Bis MPA), 2,2 dimethoxypropane, para-toluenesulfonic acid monohydrate (PTSA.H₂O), magnesium sulfate (MgSO₄), N,N′-dicyclohexylcarbodiimide (DCC), octanol, 4-dimethylaminopyridine (DMAP), pyridine, sodium carbonate (Na₂CO₃), sodium bisulfate (NaHSO₄), DOWEX 50W, 3-(4,5-dimethylthiazol-2-yl)-2,5-diphenyltetrazolium bromide (MTT), and oxalyl chloride were purchased from Sigma Aldrich. Ultra-pure lipopolysaccharide (LPS) was purchased from Invivogen, CA, USA. Slide-A-Lyzer mini dialysis devices with a 10 kDa MW cutoff was purchased from Thermo Scientific. Glutamine, penicillin/streptomycin trypsin-EDTA solution, HEPES buffer (1M), and sodium pyruvate were procured from Sigma Life Sciences. Fetal bovine serum (FBS) was purchased from Gibco Life Technologies. Acid terminated poly(DLlactide-co-glycolide) (PLGA-COOH) of inherent viscosity dL/g, 0.15 to 0.25 was purchased from Durect LACTEL® Absorbable Polymers. Interleukin (IL)-6, IL-10, and tumor necrosis factor alpha (TNF-α) cytokines were tested using BD OptEIA mouse enzyme-linked immunosorbent assay (ELISA) kits. Tween 20 was purchased from Fisher Bio-reagent. CDCl3 and DMSO-d6 were purchased from Cambridge Isotope Laboratories Inc. Regenerative cellulose membrane Amicon ultra centrifugal 100 kDa filters were purchased from Merck Millipore Ltd.

Instruments:

1H and 13C spectra were recorded on 400 MHz Varian NMR spectrometer. Electrospray ionization mass spectrometry (ESI-MS) and high-resolution mass spectrometry (HRMS)-ESI were recorded on Perkin Elmer SCIEX API 1 plus and Thermo scientific ORBITRAP ELITE instruments, respectively. Distilled water was purified by passage through a Millipore Milli-Q Biocel water purification system (18.2 MΩ) containing a 0.22 μm filter. Highperformance liquid chromatography (HPLC) analyses were made on an Agilent 1200 series instrument equipped with a multi-wavelength UV-visible and a fluorescence detector. Transmission electron microscopy (TEM) images were acquired using a Philips/FEI Tecnai 20 microscope. Gel permeation chromatographic (GPC) analyses were performed on Shimadzu LC20-AD prominence liquid chromatographer equipped with a refractive index detector and Waters columns; molecular weights were calculated using a conventional calibration curve constructed from narrow polystyrene standards using tetrahydrofuran (THF) as an eluent at a temperature of 40° C. Cells were counted using Countess® automated cell counter procured from Invitrogen life technology. Plate reader analyses were performed on a Bio-Tek Synergy HT microplate reader. Dynamic light scattering (DLS) measurements were carried out using a Malvern Zetasizer Nano ZS system.

Methods.

Cell Lines and Cell Culture.

RAW 264.7 cell line was procured from the American type culture collection (ATCC). These macrophages were grown at 37° C. in 5% CO2 in Dulbecco's Modified Eagle's Medium (DMEM) supplemented with 1% L-glutamine, 1% sodium pyruvate, 1% penicillin/streptomycin, and 10% FBS. Cells were passed every 3 to 4 days and restarted from frozen stocks after 20 passages.

Synthesis and Characterization of PLGA-b-PEG-OH:

PLGA-COOH (1.0 g, 0.18 mmol; dL/g, 0.15 to 0.25), polyethylene glycol (OH-PEG3350-OH) (1.53 g, 0.512 mmol), and DMAP

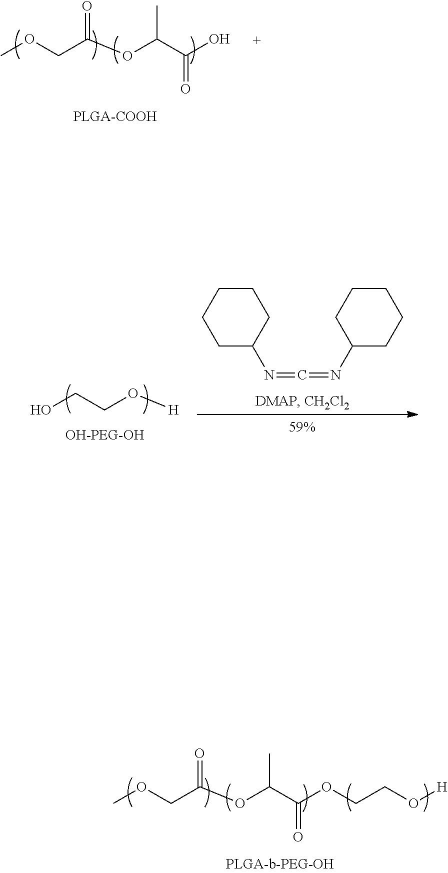

(0.02 g, 0.170 mmol) were dissolved in dry dichloromethane and stirred for 30 min at 0° C. A solution of DCC (0.106 g, 0.512 mmol) in dichloromethane was added drop wise to the reaction mixture. The reaction mixture was stirred form 0° C. to room temperature for 18 h. Precipitated DCU by-product was filtered off and the solution was evaporated by rotavap. This residue was resuspended by sonication in ethyl acetate and remaining DCU was removed. The solvent was evaporated and the resulting residue was dissolve in 5-10 mL of dichloromethane and precipitated with 40-45 mL of 1:1 mixture of methanol:diethylether and centrifuged. This process was repeated (5×) till the supernatant becomes clear solution. The resulting residue was dried under high vacuum to get a white solid polymer. Yield 0.959 g, 59%. ₁H NMR (CDCl3, 400 MHz): δ 5.20 [m, 1H], 4.81 [m, 2H], 3.63 [s, 3H], 1.56 [s, 3H] ppm. ₁₃C NMR (CDCl3, 100 MHz): δ 169.22, 166.31, 70.55, 69.01, 60.79, 16.66 ppm.

Synthesis of TPP-Hexanoic Acid:

Bromohexanoic acid (0.600 g, 3.076 mmol) and triphenylphosphine (0.968 g 3.691 mmol) were dissolved in 40 mL of acetonitrile.

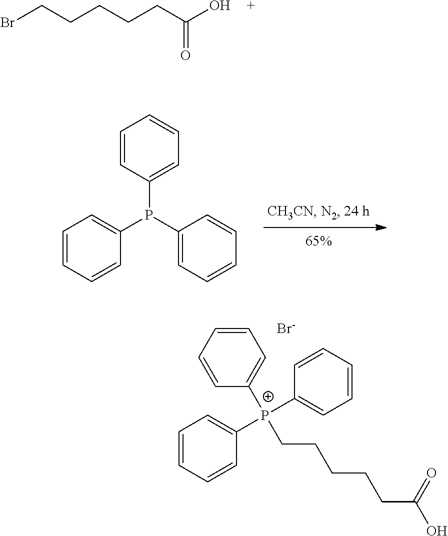 [SMILES: O=C(O)CCCCCBr ; c1ccc(P(c2ccccc2)c2ccccc2)cc1 ; [Br-] ; O=C(O)CCCCC[P+](c1ccccc1)(c1ccccc1)c1ccccc1]

This reaction was refluxed for 24 h under a N₂ environment. After 24 h, the solvent was evaporated to yield an oil, which was then precipitated with diethyl ether. The precipitate was filtered through a glass frit filter, and washed several times with diethyl ether to remove any impurities from the starting materials. The product was kept on vacuum for 1 h. Yield 0.760 g 65%. ₁H NMR (CDCl₃, 400 MHz): δ 7.78 [m, 15H], 3.56 [m, 2H], 2.44 [m, 2H], 1.68 [m, 6H] ppm.

Synthesis of PLGA-b-PEG-TPP:

TPP-hexanoic acid (0.5 g, 0.06 mmol), PLGA-b-PEG-OH (0.2 mg, 0.47 mmol), and DMAP (0.02 g, 0.17 mmol) were dissolved in dry dichloromethane and stirred for 30 min at 0° C. A solution of DCC (0.035 g, 0.170 mmol) in dichloromethane was added drop wise to the reaction mixture. This reaction mixture was stirred form 0° C. to room temperature for 18 h. The precipitated DCU by-product was filtered off and the solution was evaporated by rotavap to concentrate the volume to ˜5 mL. The concentrated solution was then precipitated with 40-45 mL of cold diethyl ether and was centrifuged at 5000 RPM at 4° C. for 5 min. The resulting supernatant was decanted and the pellet was dissolved in 2-3 mL of CH₂Cl₂ and 5 mL of methanol, 40 mL

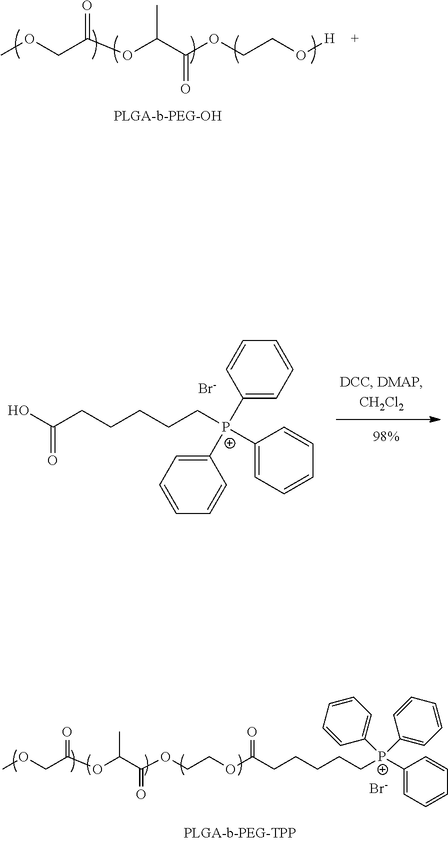

of diethyl ether was added to precipitate the product, and then centrifuged at the above settings. This process was repeated 3 times and the resulting pellet was lyophilized overnight to yield a white solid. Yield: 0.5 g, 96%. ₁H NMR (CDCl3, 400 MHz): δ 7.81 [m, 15H], 5.20 [m, 35H], 4.81 [m, 74H], 3.63 [s, 114H], 1.57 [m, 136H] ppm. ₁₃C NMR (CDCl3, 400 MHz): δ 169.23, 166.33, 134.94, 133.77, 130.38, 118.82, 117.96, 70.54, 69.00, 60.80, 16.66 ppm.

T-Asp- and NT-Asp-NP Synthesis:

Aspirin encapsulated targeted and non-targeted NPs were synthesized using the nanoprecipication method. Briefly, 100 μL from a 50 mg/mL CH₃CN solution of PLGA-b-PEG-TPP for targeted or PLGA-b-PEG-OH polymer for nontargeted NPs, and 100 μL of a 10 mg/mL CH₃CN solution of aspirin were added to 800 μL of CH₃CN. This 1 mL solution was then added drop wise to 10 mL of vigorously stirring water and was allowed to stir for 2 h. The NPs formed were then filtered using Amicon filters with a molecular weight cut off of 100 kDa, washed three times with nanopure water, and the NPs were resuspended in nanopure water at a concentration of 5 mg/mL. The size and surface charge of the NPs were characterized using dynamic light scattering method.

Synthesis and Characterization of Acetonide Protected 2,2 bis(methoxy)propionic acid (bis-MPA) (1):

2,2 bis(methoxy)propionic acid (10.0 g, 0.07 mol), 2,2 dimethoxypropane (11.6 g, 0.11 mol, 0.85 g/mL) and paratoluenesulfonic acid mono hydrate (PTSA.H₂O) (0.70 g, 0.004 mol) were dissolved in 40 mL of acetone.

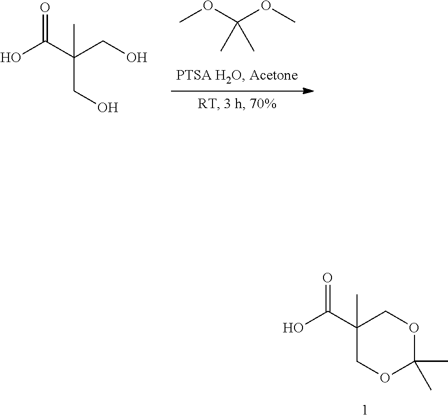

The reaction mixture was stirred at room temperature (RT) for 3 h. After 3 h, 3 mL of a 50:50 solution (by volume) of ammonia and ethanol was added to the reaction mixture to neutralize the PTSA. The solvent was evaporated and the resulting product was dissolved in 200 mL of CH₂Cl₂. This solution was then washed twice with 20 mL of nanopure water, followed by washing three washes with 40 mL of brine. The resulting solution was dried using magnesium sulfate (MgSO₄), which was then filtered out using a glass filter. The remaining CH₂Cl₂ was then evaporated and the final product was isolated as a white solid. Yield 9.0 g, (70%). ₁H NMR (CDCl₃, 400 MHz): δ 4.14 [d, 2H], 3.71 [d, 2H], 1.42 [d, 6H], 1.19 [s, 3H] ppm.

Synthesis and Characterization of Protected Bis-MPA Anhydride (2):

The acetonide protected 1 (4.22 g, 0.242 mol) was dissolved in CH₂Cl₂ (25 mL) in a round bottom flask.

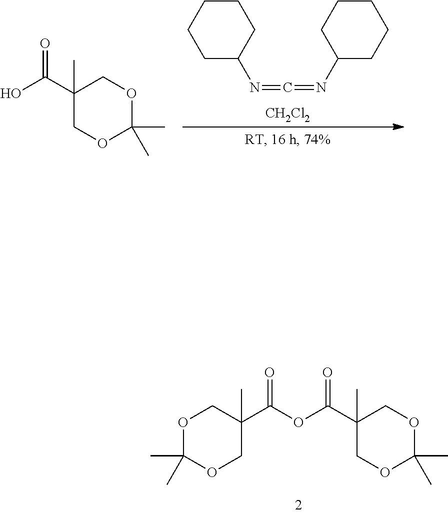

The solution was then chilled to 0° C. using an ice bath. DCC (3.2 g, 0.016 mol) was then dissolved in a separate vial in 10 mL of CH₂Cl₂ and then added dropwise to the CH₂Cl₂ solution of 1. The reaction mixture was then stirred overnight at room temperature. A white precipitate of dicyclohexylurea (DCU) was formed as a byproduct. DCU was filtered out using a glass frit filter and solvent was evaporated using a rotovap to yield an oil as the final product. The final product was placed on high vacuum for drying. Yield 4.0 g (74%). ₁H NMR (CDCl₃, 400 MHz): δ 4.17 [d, 4H], 3.68 [d, 4H], 1.41 [d, 12H], 1.21 [s, 6H] ppm.

Synthesis and Characterization of Oc-[G1]-An (3):

Octanol (2.9 g, 0.023 mol, 0.842 g/mL), DMAP (0.42 g, 0.0034 mol), and pyridine (5.4 g, 0.07 mol, 0.98 g/mL) were dissolved in 40 mL of CH₂Cl₂ in a round bottom flask and stirred constantly.

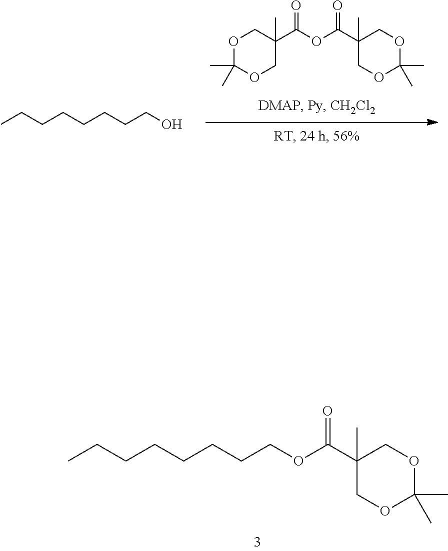

The anhydride 2 (9.0 g, 0.027 mol) was then added slowly. The reaction mixture was then stirred over night at room temperature under nitrogen. The following day, 3 mL of nanopure water was added and stirred for 20 min, and then 200 mL of CH₂Cl₂ was added. The resulting solution was washed three times with 100 mL of 10% Na2CO₃, three times with 100 mL of 10% NaHSO₄ and three times with 100 mL of brine. The resulting solution was dried over MgSO₄. This was then filtered using a glass filter, and remaining solvent was evaporated. The crude product was purified by silica flash chromatography (silica packed with hexanes) using ethylacetate:hexanes (5:95) solvent gradient to yield an oil as a product. Yield 3.7 g (56%). ₁H NMR (CDCl₃, 400 MHz): δ 4.16 [d, 2H], 4.12 [t, 2H], 3.65 [d, 2H], 1.63 [q, 2H], 1.42 [d, 3H], 1.38 [s, 3H], 1.26 [m, 10H], 1.19 [s, 3H], 0.87 [t, 3H] ppm. 13C NMR (CDCl₃, 100 MHz): δ 174.27, 98.00, 68.79, 66.01, 64.97, 48.99, 41.74, 31.75, 30.94, 29.14, 25.80, 22.62, 17.12, 14.08 ppm. HRMS-ESI (m/z): [M+Na]+ Calcd. For C₁₆H₃₀NaO₄ 309.2042. found 309.2048.

Synthesis and characterization of Oc-[G1]-(OH)2 (4):

Compound 3 (3.7 g, 0.012 mol) was dissolved in 50 mL of methanol in a round bottom flask and heated to 40° C.

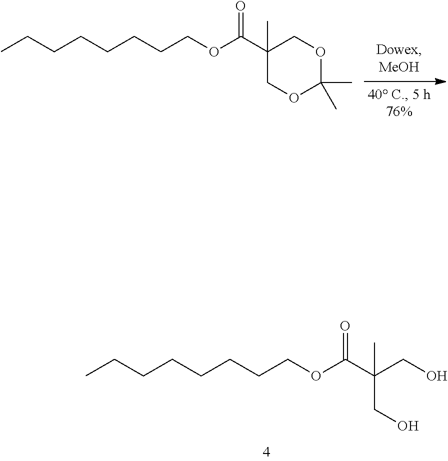

To this mixture, Dowex resin (3.5 g) was slowly added and the solution was stirred for 5 h at 40° C. The final solution was filtered through a glass frit. The methanol was then completely evaporated using a rotavap. The resulting oil was dissolved in CH₂Cl₂. This solution was filtered through MgSO₄ and the remaining solvent was evaporated using a rotovap to yield 4 as the final product. The final product was placed on high vacuum for further drying. Yield 2.4 g, 76%. ₁H NMR (CDCl₃, 400 MHz): δ 4.15 [t, 2H], 3.89 [d, 2H], 3.72 [d, 2H], 2.25 [s, 2H] 1.65 [t, 2H], 1.26 [m, 10H], 1.05 [s, 3H], 0.87 [t, 3H] ppm. 13C NMR (CDCl₃, 100 MHz): δ 175.96, 67.40, 65.14, 49.15, 31.71, 29.11, 28.45, 25.80, 22.58, 17.13, 14.03 ppm. HRMS-ESI (m/z): [M+H]+ Calcd. for C₁₃H₂₇O₄ 247.1909. found 247.1902.

Synthesis and Characterization of Aspirin Acid Chloride (5):

Acetylsalicylic acid (2.2 g, 0.0122 mol) and oxalyl chloride (3.1 g, 0.0244 mol, 1.5 g/mL) were dissolved in 50 mL of CH₂Cl₂ in a 100 mL round bottom flask. Few drops of DMF were added to catalyze the reaction.

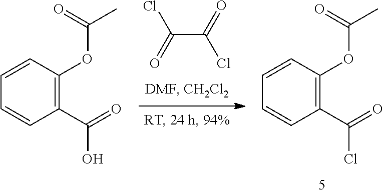

The reaction mixture was then stirred overnight at room temperature. The solvent was then evaporated to yield 5 as a yellow oil. Yield 2.2 g, 94%. ₁H NMR (CDCl3, 400 MHz): δ 8.23 [d, 1H], 7.69 [t, 1H], 7.42 [t, 1H], 7.18 [d, 1H], 2.36 [s, 3H] ppm. ₁₃C NMR (CDCl₃, 100 MHz): δ 169.18, 164.64, 150.33, 136.06, 134.39, 132.48, 126.47, 124.24, 20.87 ppm. HRMS-ESI (m/z): [M−H]− calcd. for C₉H₆ClO₃ 197.5940. found, 197.8038.

Synthesis and Characterization of Oc-[G1]-(Asp)2 (6):

Compound 4 (0.5 g, 0.00204 mol), DMAP (7.5 mg, 0.0006 mol), and pyridine (1.3 g, 0.016 mol, 0.98 g/mL) were dissolved in 50 mL of CH₂Cl₂. Compound 5 (1.6 g, 0.008 mol) was added drop-wise. The reaction mixture was stirred overnight at room temperature under N₂ flow. The following day, 3 mL of nanopure water was added, followed by the addition of 40 mL of CH2Cl2. The solution was then washed three times with 50 mL of 1 M NaHCO₃, three times with 50 mL of 10% NaHSO4, and three times with 50 mL of brine. The final solution was dried over MgSO₄ to remove any remaining water. The solvent was then evaporated to yield an oil. The crude product was then purified using silica flash column chromatography (silica packed with hexanes) using ethylacetate:hexane (10:90) and then ethylacetate:hexane (15:85), and the product was isolated as a pale yellow oil. Yield: 947 mg, 87%. ₁H NMR (CDCl3, 400 MHz): δ 7.92 [d, 2H], 7.56 [t, 2H], 7.28 [t, 2H], 7.11 [d, 2H], 4.49 [s, 3H], 4.13 [t, 2H], 2.32 [s, 5H], 1.58 [q, 1H], 1.37 [s, 2H], 1.18 [m, 10H], 0.85 [t, 3H] ppm. 13C NMR (CDCl3, 100 MHz): δ 1172.61, 69.62, 163.51, 151.01, 134.09, 131.45, 126.01, 123.90, 122.55, 66.15, 65.57, 46.60, 31.74, 29.11, 29.05, 28.5

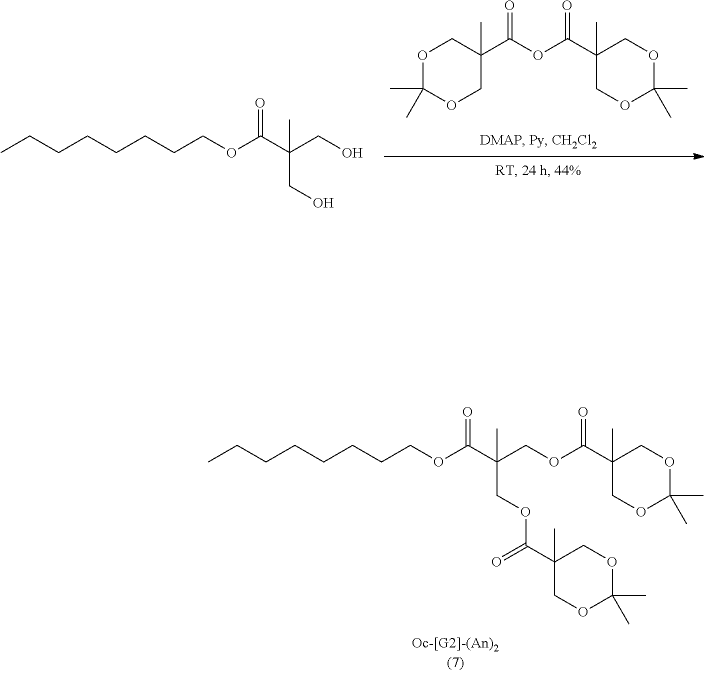

0, 25.80, 22.59, 21.02, 20.98, 17.97, 14.07 ppm. HRMS-ESI (m/z): [M+Na]+ calcd. for C₃₁H₃₉NaO₁₀ 593.2363. found 593.2354.

Synthesis and Characterization of Oc-[G2]-(an)₂ (7):

Compound 4 (1.8 g, 0.007 mol), DMAP (0.269 g, 0.0022 mol) and pyridine (4.7 g, 0.06 mol, 0.98 g/mL) were dissolved in 60 mL of CH₂Cl₂ in a round bottom flask and stirred constantly. Compound 2 (6.3 g, 0.02 mol) was added drop-wise into the stirring solution. This reaction mixture was stirred over night at room temperature under constant N₂ flow. The following day, 3 mL of nanopure water was added; followed by the addition of 200 mL of CH₂Cl₂. The solution was then washed three times with 50 mL of 1 M NaHCO₃, three times with 50 mL of 10% NaHSO₄ and three times with 50 mL of brine. The final solution was dried over MgSO₄ to remove any remaining water. The solvent was then evaporated to yield an oil. The crude product was then purified using silica flash column chromatography (silica packed with hexanes) using ethylacetate:hexane (10:90), then ethylacetate:hexane

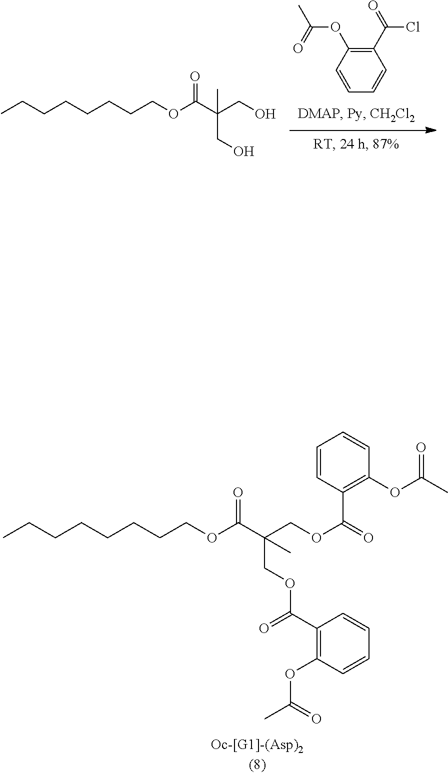

(20:80), and the product was isolated as an oil. Yield: 1.83 g, 44%. ₁H NMR (CDCl₃, 400 MHz): δ 4.31 [s, 4H], 4.13 [d, 4H], 4.10 [t, 2H], 3.63 [d, 4H], 1.62 [q, 2H], 1.41 [s, 6H], 1.35 [s, 6H], 1.27 [m, 12H], 1.15 [s, 6H], 0.87 [t, 3H] ppm. 13C NMR (CDCl₃, 100 MHz): δ 173.51, 172.59, 98.07, 65.95, 65.90, 65.46, 65.33, 46.68, 41.99, 31.75, 29.16, 29.13, 28.50, 25.85, 24.80, 22.61, 22.37, 18.54, 17.75, 14.06 ppm. HRMS-ESI (m/z): [M+Na] calc. for C₂₉H_{SO}NaO₁₀ 581.3302. found 581.3293.

Synthesis and characterization of Oc-[G2]-(OH)4 (8):

Compound 7 (1.83 g, 0.003 mol) was dissolved in 70 mL of methanol in a round bottom flask and the reaction mixture was heated to 40° C. Dowex resin (8 g) was slowly added to the mixture. This solution was stirred for 5 h at 40° C. The final solution was then filtered through a glass frit, and the solvent was evaporated completely to yield a red oil.

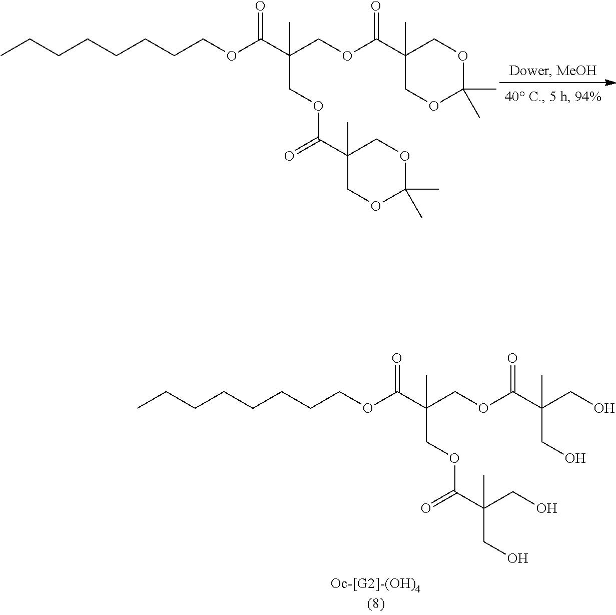

This oil was then dissolved in 40 mL of CH2Cl2 and filtered through MgSO4. The remaining solvent was then evaporated to yield a light orange solid. The final product was placed on high vacuum for drying. Yield: 1.5 g, 94%. ₁H NMR (CDCl₃, 400 MHz): δ 4.43 [d, 2H], 4.28 [d, 2H], 4.13 [t, 2H], 3.82 [t, 4H], 3.70 [t, 4H], 3.20 [s, 4H], 1.62 [q, 2H], 1.30 [s, 3H], 1.26 [m, 10H], 1.03 [s, 6H], 0.87 [t, 3H] ppm. 13C NMR (CDCl₃, 100 MHz): 175.13, 173.01, 68.19, 65.68, 64.84, 49.62, 46.31, 31.74, 29.13, 28.47, 25.82, 22.61, 18.16, 17.09, 14.07 ppm. HRMS-ESI (m/z): [M+H] calc. for C₂₃H₄₃O₁₀ 479.2856. found 479.2862.

Synthesis and Characterization of Oc-[G2]-(Asp)4 (9):

Compound 8 (1 g, 0.002 mol), DMAP (153 mg, 0.0013 mol), and pyridine (2.7 g, 0.034 mol, 0.98 g/mL) were dissolved in 80 mL of CH₂Cl₂ and stirred at room temperature. Compound 5 (2.4 g, 0.011 mol) was then added drop-wise to the stirring solution. The reaction mixture was stirred overnight under N₂ flow. The next day, 10 mL of CH₂Cl₂ was added. 10 min later, 3 mL of

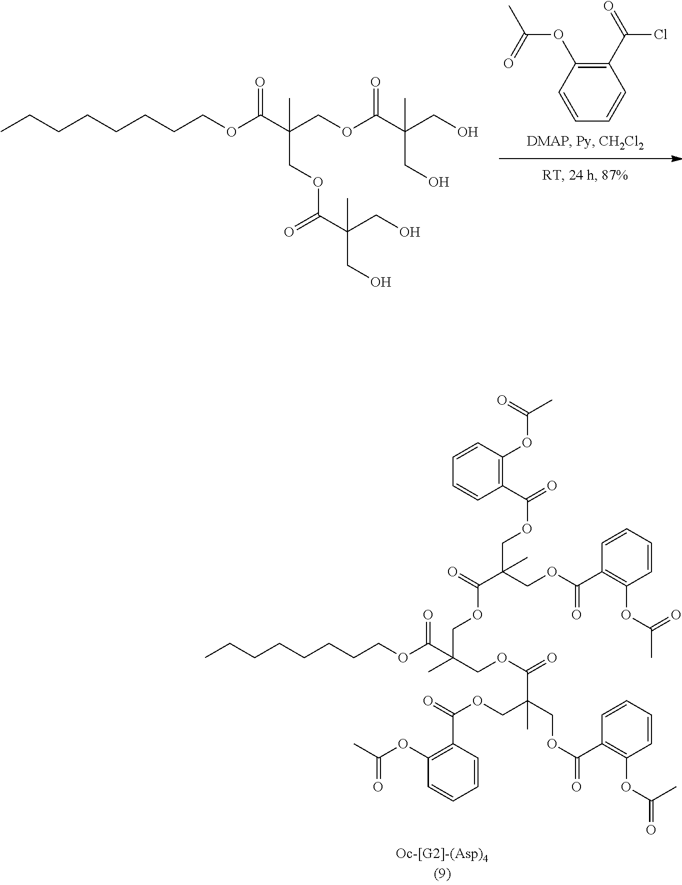

nanopure water was added, and the resulting solution was washed three times with 50 mL of 1 M NaHCO₃, three times with 50 mL of 10% NaHSO₄ and three times with 50 mL of brine. The solution was then dried over MgSO₄, and the solvent was evaporated to yield a dark brown oil. The crude product was then purified using silica flash column chromatography (silica packed with hexanes) using ethylacetate:hexane (5:95) followed by ethylacetate:hexane (10:90), ethylacetate:hexane (15:85), ethylacetate:hexane (20:80), ethylacetate:hexane (25:75) and finally ethylacetate:hexane (30:70). The separate fractions were concentrated and the purity of the concentrated product was checked by thin layer chromatography (TLC). The product showed two spots on TLC. This crude product was then purified using silica flash column (packed with CH2Cl2) initially with methanol:dichloromethane (0.5:99.5) and then methanol:dichloromethane (1:99). The solvent was evaporated to yield a pale yellow oil. Yield 1.2 g, 87%. 1H NMR (CDCl3, 400 MHz): δ 7.90 [d, 4H], 7.54 [t, 4H], 7.27 [t, 4H], 7.09 [d, 4H], 4.44 [m, 8H], 4.26 [m, 4H], 3.67 [t, 2H], 2.30 [s, 12H], 1.51 [q, 2H], 1.30 [s, 6H], 1.22 [m, 10H], 1.15 [m, 3H], 0.85 [t, 3H] ppm. 13C NMR (CDCl3, 400 MHz): δ 172.07, 171.82, 169.57, 163.40, 150.97, 134.13, 131.44, 126.06, 123.88, 122.46, 70.55, 65.79, 53.42, 46.66, 46.51, 31.74, 29.12, 28.41, 25.73, 22.60, 20.97, 17.84, 17.47, 14.08 ppm HRMS-ESI (m/z): [M+Na] calc. for C59H66NaO22 1149.3943. found 1149.3945.

T-(Asp)₄- and NT-(Asp)₄-NP Synthesis:

T-(Asp)₄- and NT-(Asp)₄-NPs were synthesized using the nanoprecipication method. Briefly, 100 μL from a 50 mg/mL CH3CN solution of PLGA-b-PEG-OH for NT-NPs or PLGA-b-PEG-TPP for T-NPs was mixed with 100 μL of a 10 mg/mL solution of Oc-[G2]-(Asp)₄ in CH3CN and this solution was then diluted to 1 mL using CH3CN. This 1 mL solution containing the polymer and aspirin analogues was added drop wise to 10 mL of vigorously stirring water and was allowed to stir for 2 h. These solutions were then filtered using Amicon filters with a molecular weight cut off of 100 kDa, washed three times with nanopure water, and the NPs were resuspended in nanopure water at a concentration of 5 mg/mL. Diameter (Zaverage) and surface charge of the NPs were determined using 0.5 mg/mL NP suspension in water on a Malvern Zetasizer. Characterization of NPs by TEM was conducted on samples by mixing 50 μg/mL NP suspension with 2% weight/volume uranyl acetate in nanopure water and depositing this mixture on a carbon coated copper grid (Cat. No. 71150, CF300-Cu, Electron Microscopy Science, Hatfield, Pa.). After drop casting, water evaporated by drying the grid overnight at room temperature.

T-(Asp)2- and NT-(Asp)2-NP Synthesis:

These NPs were synthesized following methods mentioned above for T/NT-(Asp)4-NPs using Oc-[G1]-(Asp)2 instead Oc[G2]-(Asp)4.

Aspirin Quantification in NPs:

The amount of aspirin encapsulated in the synthesized NPs was quantified using HPLC. To create the standard curves for aspirin, Oc-[G1]-(Asp)₂, or Oc-[G2]-(Asp)₄, 800 μL of a 1 mg/mL solution was created, and then serial dilutions were done to create 400 μL solutions with concentration of 1000 μg/mL, 500 μg/mL, 250 μg/mL, 125 μg/mL, 62.5 μg/mL, 31.25 μg/mL, 15.625 μg/mL, and 7.8125 μg/mL. To these solutions, 100 μL of 0.1 M NaOH solution and 100 μL of water were added for a total volume of 600 μL. To prepare the samples for analysis, 400 μL of CH3CN, 100 μL of 0.1 M NaOH, and 100 μL of the synthesized NPs were added. These solutions were then incubated at 37° C., for 24 h, and then analyzed using an Agilent 1260 Infinity series HPLC system. The mobile phase used was 50:50 CH3CN:water. Aspirin was converted to salicylic acid, which produces a peak at approximately 12.5 min at 295 nm wavelength. The peak areas of the samples were obtained and using the prepared standard curve the aspirin concentration in the NPs were calculated.

Release of Aspirin from T-(Asp)₄- and NT-(Asp)₄-NPs:

To assess the release of aspirin from the NPs, 800 μL of each 5 mg/mL NP solution was diluted to 2.4 mL with nanopure water. Then, 100 μL of this solution was added to 24 Thermo Scientific Slide-A-Lyzer MINI dialysis tubes. These tubes were then floated in a bath of 1×PBS with gentle shaking at 37° C. for 120 h. For the first 12 h of incubation, the PBS bath was changed every 3 hours. After that point, the bath was changed every 12 hours. For each nanoparticle type, two of these tubes were collected from the bath at time points of 0, 1, 2, 4, 6, 8, 12, 24, 48, 72, 96, and 120 h after the beginning of incubation. The solution in the tubes was collected in 1.5 mL microcentrifuge tubes, diluted to 500 μL to make volumes uniform, and stored at 4° C. Once all tubes were collected in this way, 100 μL of the collected solutions was added to 400 μL of CH3CN and 100 μL of 0.1 M NaOH. These were incubated at 37° C., along with standard samples prepared as in the aspirin Quantification procedure, and then analyzed with the Agilent 1260 Infinity series HPLC system. The concentrations of aspirin determined through this analysis were then used to calculate the percent of the aspirin mass that had been released at each time point.

Cytotoxicity Assay in RAW 264.7 Cells:

Toxicity of T/NT-(Asp)₂/(Asp)₄-NPs was studied in RAW 264.7 macrophages by using the well-known MTT-based colorimetric assay. RAW 264.7 cells (3000 cells/well/100 μL) were seeded on a 96 well plate and allowed to grow 24 h at 37° C. in 5% CO2. Next day, media in each well was removed and replenished with 100 μL of fresh media. All four types of NPs were added in increasing concentrations and each concentration had three replicates. After 24 h of incubation with the NPs, media was again changed and fresh media was added to each well. Following 48 h of further incubation, MTT was added (5 mg/mL, 20 [symbol] L/well), and the plates were incubated for 5 h for conversion of MTT to formazan by cellular oxidoreductase enzymes. The media was removed and lysis was carried out using 100 μL of DMSO, followed by homogenization of formazan with gentle shaking for 5 min at room temperature. The absorbance of the resultant solution in each well was read at 550 nm with a background reading at 800 nm. Cytotoxicity was expressed as mean percentage increase relative to the untreated control±standard deviation. Control values were set at 0% cytotoxicity or 100% cell viability. Cytotoxicity data (where appropriate) was fitted to a sigmoidal curve and a three parameters logistic model used to calculate the inhibitory concentration-50 (IC50) that is the concentration of test article under investigation showing 50% inhibition in comparison to untreated controls. These analyses were performed with GraphPad Prism (San Diego, U.S.A).

In Vivo Inflammation Studies:

Anti-inflammatory properties of T/NT-(Asp)₄-NPs and aspirin were evaluated in LPS stimulated mouse model. C57BL//6 types of male mice of 12 week age or BALB/c white albino male mice of 8 week age were first injected with 20 mg/kg of T/NT-(Asp)₄-NPs (these concentrations are with respect to aspirin) or 20 mg/kg of aspirin by tail vein injection. After 12 h, 100 μg of LPS/animal was administered by intraperitoneal injection to investigate whether this new aspirin analogue in NP formulation can prevent the animals from LPS induced inflammatory responses. Either 1.5 h or 3 h after LPS injection, blood samples were collected and serum was isolated by centrifugation (2400 rpm, 30 min) for analyses of pro-inflammatory IL-6 and TNF-α cytokines and IL-10 as anti-inflammatory cytokine. ELISA was carried out on the serum samples for the cytokines IL-6, TNF-α, IL-10 according to the methods reported by Marrache et al., ACS Nano, 2013, 7392-7402; and Pathak, et al., Angew. Chem. Int. Ed. Engl., 2014, 53, 1963-1967 by performing blocking of antibody coated plates using 10% FBS in PBS for 1 h at room temperature followed by 3 washes. The serum samples (20 μL) or standard were incubated on the plates for 2 h at room temperature followed by several washing steps and serial incubations with the cytokine-biotin conjugate and streptavidin working solution. ELISA was finally followed by using a colorimetric assay by adding the substrate reagent containing 3,3′,5,5′-tetramethylbenzidine (100 μL) to each well and incubation got 15 min, the reaction was then stopped by using 50 μL H2SO4 (2N). The absorbance of the product formed was recorded at 450 nm using a BioTek Synergy HT well plate reader.

Thus, embodiments of MODIFICATION OF DRUGS FOR INCORPORATION INTO NANOPARTICLES are disclosed. One skilled in the art will appreciate that the nanoparticles and methods described herein can be practiced with embodiments other than those disclosed. The disclosed embodiments are presented for purposes of illustration and not limitation.

What is claimed is:

1. An nonsteroidal anti-inflammatory drug (NSAID) prodrug of Formula I:
(T)ₙ−L−(D)ₘ (I), where
T is a lipophilic moiety;
n is a positive integer (such as 1 to 10);
D is a releasable NSAID moiety;
m is a positive integer (such as 1 to 50); and
L is a linker.

2. A NSAID prodrug according to claim 1, wherein D is a releasable aspirin moiety.

3. An NSAID prodrug according to claim 1, wherein T is a saturated or unsaturated, straight or branched chain hydrocarbon having between 4 and 20 carbons.

4. An NSAID prodrug according to claim 1, wherein T is a straight or branched chain C₆-C₂₀ alkyl.

5. An NSAID prodrug according to claim 1, wherein T is octanyl.

6. An NSAID prodrug according to claim 1, wherein n is 1.

7. An NSAID prodrug according to claim 1, wherein D is bound to L via an ester linkage.

8. An NSAID prodrug according to claim 1, wherein m is 2 or more.

9. An NSAID prodrug according to claim 1, wherein m is 4.

10. An NSAID prodrug according to claim 1, wherein L is a 2, 2, bis(methoxy)propionyl moiety or dendrimer.

11. An aspirin prodrug of Formula II:

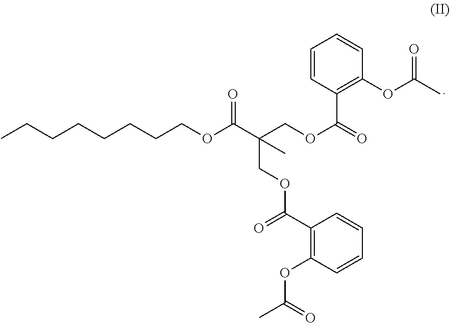 [SMILES: CCCCCCCCOC(=O)C(C)(COC(=O)c1ccccc1OC(C)=O)COC(=O)c1ccccc1OC(C)=O]

12. An aspirin prodrug of Formula III:

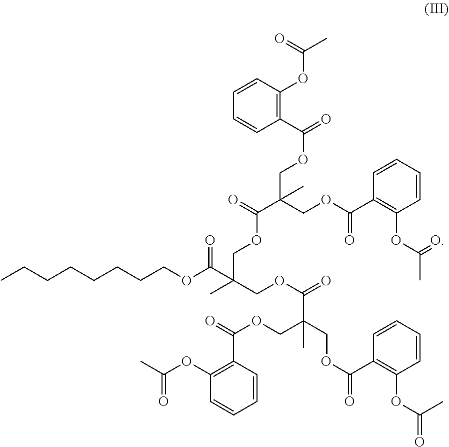 [SMILES: CCCCCCCCOC(=O)C(C)(COC(=O)C(C)(COC(=O)c1ccccc1OC(C)=O)COC(=O)c1ccccc1OC(C)=O)COC(=O)C(C)(COC(=O)c1ccccc1OC(C)=O)COC(=O)c1ccccc1OC(C)=O]

13. A nanoparticle comprising an NSAID prodrug according to claim 1.

14. A nanoparticle according to claim 13, wherein the nanoparticle comprises a hydrophobic core.

15. A nanoparticle according to claim 13, further comprising a mitochondria targeting moiety.

16. A nanoparticle according to claim 13, wherein the nanoparticle comprises a diameter of 100 nanometers or less.

17. A method for inhibiting cyclooxygenase in a patient in need thereof, comprising:
administering a nanoparticle according to claim 13 to the patient.

18. A method for treating a neurodegenerative disease in a patient in need thereof; comprising:
administering a nanoparticle according to claim 13 to the patient.
